(19) United States
(12) Patent Application Publication
(10) Pub. No.: US 2021/0046277 A1
(43) Pub. Date: Feb. 18, 2021

(54) SELF-CLEANING CATHETER SYSTEMS

(71) Applicants: MICROBOT MEDICAL LTD., Caesarea (IL); TECHNION RESEARCH & DEVELOPMENT FOUNDATION LIMITED, Haifa (IL)

(72) Inventors: Or SAMOOCHA, Bustan Hagalil (IL); Simon SHARON, Maayan Zvi (IL); Yosef PORAT, Afula (IL); Moshe SHOHAM, Hoshaya (IL); Harel GADOT, Hingham, MA (US); Idan BOADER, Karmiel (IL); Danna PERLMAN, Haifa (IL); Eyal BEN-MOSHE, Kibbutz Hulda (IL)

(21) Appl. No.: 16/965,590

(22) PCT Filed: Jan. 31, 2019
(86) PCT No.: PCT/IL2019/050126

§ 371 Date: Jul. 28, 2020

Related U.S. Application Data
(60) Provisional application No. 62/625,928, filed on Feb. 2, 2018.
(60) Provisional application No. 62/767,613, filed on Nov. 15, 2018.
(60) Provisional application No. 62/784,729, filed on Dec. 25, 2018.

(51) Int. Cl. A61M 25/00; B08B 9/04; B08B 13/00; B08B 7/02

(52) U.S. Cl. CPC A61M 25/00; B08B 9/04; A61M 2005/16863; B08B 7/02; A61M 2025/0019; B08B 13/00

(57) ABSTRACT

Disclosed is a self-cleaning catheter system for fluid passage including a catheter, configured to be implanted in a body cavity of a subject and including at least one aperture fluidly coupling the catheter to the outside thereof, a cleaning unit configured for motion in the catheter such as to at least one of mechanically prevent, remove and mitigate occlusion in the at least one aperture, and an implantable controller. The cleaning unit is functionally associated with the controller, which is configured to (i) receive at least one signal indicative of a state of occlusion in the catheter, and (ii) provide an indication of the state of occlusion at least if the at least one signal indicates a blockage in the catheter and/or (iii) activate the cleaning unit if the at least one signal indicates a blockage of the catheter.

### TECHNICAL FIELD

The present disclosure relates generally to self-cleaning catheter systems for fluid delivery, drainage, and/or passage.

### BACKGROUND

Shunts are often used as internal medical devices to drain aberrant fluids from different organs. FIG. 1A schematically depicts a prior art cerebral shunt 15 for draining cerebrospinal fluid (CSF) implanted in an infant patient 25. Shunt 15 includes a ventricular catheter 35, a drain tube 37, and a valve 39 regulating the flow of fluid from ventricular catheter 35 to drain tube 37. Ventricular catheter 35 is implanted in a brain ventricle (not indicated). FIG. 1B is a close-up view of ventricular catheter 35. A catheter head 41 of ventricular catheter 35 includes a plurality of apertures 47 and 49, along its length; the apertures often having different sizes and different spacings, such that CSF accumulated around ventricular catheter 35 drains through the apertures into drain tube 37, and away from the brain ventricle. The excess CSF is generally drained into a body cavity such as the abdomen. Ventricular catheter 35 may have length calibrations imprinted thereon, so that the surgeon can estimate how far ventricular catheter 35 has been inserted into the cranial cavity. Drain tube 37 is generally implanted just beneath the skin, with access to the cranial region to be drained, and into the abdominal cavity, being achieved by means of small incisions 55 in the meninges and the peritoneum respectively. To allow the patient to grow into adulthood without having to replace the shunt, an end section 61 of drain tube 37 may be bundled up in the abdominal cavity, so that it can unravel as the patient grows.

Such prior art simple shunts, as described above, generally have two major problems: (i) the inlet apertures might get clogged, and (ii) the ventricular catheter might become contaminated and thereby potentially cause an infection. When the ventricular catheter becomes clogged (e.g. due to clogging of the inlet apertures), an attempt to remove it from the body by surgery should be made. In cases where it is impossible to remove, another ventricular catheter may be placed in parallel to the malfunctioning one. When the ventricular catheter is contaminated it must be removed from the body by surgery. Surgeries of this kind are often high-risk procedures.

The simple prior art shunts depicted in FIGS. 1A and 1B have a significant drawback in that after some period of time inside the human body, living tissue growth may result in blockage of the apertures by the tissue. This tissue is generally the main cause of shunt blockage. When trying to withdraw the shunt by surgery, the ingrown tissue may tear, causing intraventricular bleeding, which might be life threatening.

### SUMMARY

Aspects of the disclosure, according to some embodiments thereof, relate generally to implantable, self-cleaning catheter systems for fluid delivery, drainage, and/or passage. More specifically, but not exclusively, aspects of the disclosure, according to some embodiments thereof, relate to implantable, self-cleaning catheter systems configured for monitoring physical parameters indicative of a condition of the subject (e.g. intracranial pressure when the catheter system is implanted in the brain) and/or proper functionality of the catheter system. The monitoring may be performed essentially continuously (when the catheter system includes a power source) or each time a cleaning session is initiated (e.g. at least once a day). Exceeding predetermined thresholds and/or sharp changes in the measured values of the physical parameters may indicate that medical intervention is required. Trend analysis of the measured values may advantageously allow one to predict in advance the development of a physical condition (which may require medical attention).

Aspects of the disclosure, according to some embodiments thereof, relate to implantable, self-cleaning catheter systems configured for activation according to a fixed schedule, either manually (i.e. by the subject or a caretaker) or automatically (i.e. self-activation according to a pre-programmed schedule).

Aspects of the disclosure, according to some embodiments thereof, relate to implantable, self-cleaning catheter systems configured for self-activation on receipt of a signal indicative of occlusion in the catheter system (i.e. “closed-loop” systems). Advantageously, cleaning sessions of the catheter systems may be performed as necessity dictates, rather than according to a fixed schedule. Consequently, fewer cleaning sessions may be performed on average, so that, in embodiments including an implantable power source (e.g. an implantable battery), the lifetime of the power source (and the time between recharging and/or replacements thereof), may potentially be increased.

Further aspects of the disclosure, according to some embodiments thereof, relate to wearable external activation units configured to launch and power, through wireless power transfer (WPT), a cleaning session effected by the self-cleaning catheter systems. Advantageously, a wearable external activation unit facilitates the activation of a cleaning session, and may potentially, particularly when the subject is an infant, increase compliance of the subject with the treatment schedule.

Still further aspects of the disclosure, according to some embodiments thereof, relate to smartphone applications (apps) configured to allow the subject or a caretaker thereof to operate the catheter system and launch a cleaning session. Advantageously, an app offers a convenient graphical user interface for operating the catheter system. Further, according to some embodiments, the app may be configured to estimate the time for a next cleaning session based on occlusion data received in real-time, optionally employing trend analysis based on “historical” occlusion data (i.e. past occlusion data received, such as occlusion data obtained before prior cleaning sessions).

Thus, according to an aspect of some embodiments, there is provided a self-cleaning catheter system for fluid passage. The catheter system includes:
A catheter, configured to be implanted in a body cavity of a subject, the catheter including at least one aperture fluidly coupling the catheter to the outside thereof.
A cleaning unit configured for motion in the catheter such as to at least one of mechanically prevent, remove and mitigate occlusion in the at least one aperture.
An implantable controller (e.g. micro-controller).

The cleaning unit is functionally associated with the implantable controller. The implantable controller is configured to receive at least one signal indicative of a state of occlusion in the catheter, and, if the at least one signal indicates at least a partial blockage in the catheter, provide an indication of the state of occlusion and/or activate the cleaning unit.

According to some embodiments, the implantable controller is configured to provide the indication of the state of occlusion also when the at least one signal indicates no blockage in the catheter.

According to some embodiments, the catheter includes a catheter tip member, which is distally positioned and includes one or more of the at least one aperture.

According to some embodiments, the body cavity includes a ventricle.

According to some embodiments, the received at least one signal includes a pressure-related signal indicative of a pressure in at least one of the body cavity and the catheter.

According to some embodiments, the pressure being above an upper pressure threshold is indicative of at least partial blockage in the catheter.

According to some embodiments, the ventricle includes a brain ventricle and the pressure-related signal is indicative of intracranial pressure.

According to some embodiments, the implantable controller is configured to be implanted in the head of the subject outside the skull and beneath the skin.

According to some embodiments, the received at least one signal includes a fluid flow-related signal indicative of a fluid flow rate through the catheter.

According to some embodiments, the fluid flow rate being below a flow rate threshold is indicative of at least partial blockage in the catheter.

According to some embodiments, the implantable controller is configured to assess the state of occlusion based, at least in part, on the at least one received signal.

According to some embodiments, the catheter is fluidly connected to a valve and/or a pump for evacuating fluid from the catheter. The valve and/or the pump are functionally associated with the implantable controller, which is configured to open/close the valve and/or switch on/off the pump.

According to some embodiments, the catheter system further includes at least one sensor, which is implantable and communicatively associated with the implantable controller.

The at least one signal, received by the implantable controller, may be sent/generated by the at least one sensor.

According to some embodiments, the at least one sensor is configured to be automatically activated on a periodic basis.

According to some embodiments, the at least one sensor is configured for continuous or substantially continuous monitoring.

According to some embodiments, the at least one sensor is housed in the catheter or embedded in walls of the catheter.

According to some embodiments, one or more of the at least one sensor is housed in the catheter tip member.

According to some embodiments, the at least one sensor includes a pressure sensor configured to measure the pressure within the catheter and/or the body cavity.

According to some embodiments, the at least one sensor includes a flowmeter configured to measure the fluid-flow rate (or, more generally, fluid flow related parameters) in the catheter.

According to some embodiments, the at least one sensor includes an additional sensor positioned in, on, or near the valve and/or the pump. The additional sensor may be configured to measure pressure and/or fluid-flow rate.

According to some embodiments, the catheter system further includes an implantable power receiver configured for wireless power transfer (WPT) from an external activation unit. The implantable power receiver is further configured to at least partially power the catheter system.

According to some embodiments, the implantable controller and the implantable power receiver are both housed in an implantable casing.

According to some embodiments, the implantable power receiver may include a coil of conducting wire and may be configured for WPT based on inductive coupling.

According to some embodiments, the implantable power receiver may further be configured to transmit the state of occlusion indication (i.e. the indication of the state of occlusion) to the external activation unit. The external activation unit may further be configured to trigger an alert when the state of occlusion indication indicates at least partial blockage in the catheter.

According to some embodiments, the implantable controller includes a communication unit configured to transmit the state of occlusion indication to the external activation unit. The external activation unit may be configured to trigger an alert when the state of occlusion indication indicates at least partial blockage in the catheter.

According to some embodiments, the external activation unit includes a processing circuitry (e.g. a computer processor(s) and non-transient memory) configured to assess whether the catheter is at least partially blocked based, at least in part, on the state of occlusion indication.

According to some embodiments, the external activation unit is wearable.

According to some embodiments, the body cavity includes a brain ventricle and the external activation unit is a headpiece or is configured to be mounted on a headpiece.

According to some embodiments, the external activation unit includes a user interface configured to generate the alert and to allow the subject and/or a caretaker thereof to activate/operate the cleaning unit.

According to some embodiments, the implantable controller is configured to prevent the activation of the cleaning unit if the power received by the implantable power receiver is above an upper power threshold.

According to some embodiments, the implantable controller is configured to induce an electrical disconnection between the implantable power receiver and the cleaning unit if the power received by the implantable power receiver exceeds an upper power threshold.

According to some embodiments, the implantable controller is configured to prevent the activation of the cleaning unit if the power received by the implantable power receiver is below a lower power threshold.

According to some embodiments, the implantable controller is configured to prevent activation of the cleaning unit if the power, received by the implantable power receiver, does not originate from the external activation unit.

According to some embodiments, the implantable controller is further configured to perform an automatic shutdown procedure when the duration of the WPT exceeds an upper time threshold.

According to some embodiments, the external activation unit is communicatively associated with a mobile communication device.

According to some embodiments, the mobile communication device includes at least one of a smartphone, a smartwatch, a tablet, and a laptop.

According to some embodiments, the external activation unit is further configured to be operated/controlled using software installable on the mobile communication device.

According to some embodiments, the software is configured to allow a user to operate/control the external activation unit (and thereby the catheter system), using a user interface of the mobile communication device.

According to some embodiments, the catheter system further includes an implantable power source (e.g. battery) configured for at least partially powering the catheter system.

According to some embodiments, the implantable controller and the implantable power source are both housed in an implantable casing.

According to some embodiments, the implantable controller includes a communication unit configured to transmit the state of occlusion indication to an external controller. The external controller may be configured to generate an alert when the state of occlusion indication indicates at least partial blockage in the catheter.

According to some embodiments, the external controller includes a processing circuitry configured to determine a degree of blockage in the catheter and/or a time for next cleaning session.

According to some embodiments, the implantable controller is configured to be operated/controlled using software installable on the external controller.

According to some embodiments, the software is configured to allow a user to operate/control the implantable controller (and thereby the catheter system), using a user interface of the external controller.

According to some embodiments, the external controller is a mobile communication device.

According to some embodiments, the implantable power source (e.g. battery) is rechargeable and configured to be recharged by WPT.

According to some embodiments, the alert signals that a cleaning session is required.

According to some embodiments, the catheter includes a catheter tube fluidly connected to the catheter tip member. The catheter tip member at least partially houses the cleaning unit.

According to some embodiments, the cleaning unit includes an elongated shaft including at least one arm configured to project into the at least one aperture and to move therein.

The movement of the at least one arm may prevent at least tissue from entering at least some of the at least one aperture when the catheter tip member is implanted within the body cavity.

According to some embodiments, the cleaning unit is configured to allow vibration thereof, such that the motion of the cleaning unit in the catheter includes vibration. The movement of the at least one arm within the at least one aperture may be induced by the vibration of the cleaning unit.

According to some embodiments, the catheter system further includes a motion generator (e.g. a vibration generator) functionally associated with the implantable controller and configured to induce the motion (e.g. vibration) of the cleaning unit.

According to some embodiments, the motion generator (e.g. vibration generator) is an electromagnet. The cleaning unit includes, or is mechanically coupled to, a magnet of the electromagnet.

According to an aspect of some embodiments, there is provided an external activation unit for powering an implantable, self-cleaning catheter system for fluid passage. The external activation unit includes a power transmitter and a processing circuitry functionally associated with the power transmitter. The power transmitter is configured for wireless power transfer (WPT) to an implantable power receiver of the catheter system when the catheter system is implanted in a body cavity of a subject. The power receiver is configured to power a cleaning session of the catheter system. The cleaning session is configured to prevent, remove, and/or mitigate occlusion in the catheter system.

According to some embodiments, the catheter system may include a catheter, configured to be implanted in the body cavity of a subject, a cleaning unit configured to mechanically prevent, remove, and/or mitigate blockage in the catheter, and an implantable controller (e.g. micro-controller). The cleaning unit may be functionally associated with the implantable controller, which is configured at least to activate the cleaning unit.

According to some embodiments, the catheter system is configured for at least one draining fluid from the body cavity and delivering fluid into the body cavity.

According to some embodiments, the external activation unit is further configured for placement on, to be attached to, worn on, and/or to be held against a body part of the subject, such as to enable the WPT from the power transmitter to the implantable power receiver. The body part includes the body cavity.

According to some embodiments, the WPT is based on inductive coupling between the implantable power receiver and the power transmitter.

According to some embodiments, the WPT is based on capacitive coupling between the implantable power receiver and the power transmitter.

According to some embodiments, the body cavity includes a brain ventricle, the fluid includes cerebrospinal fluid, and the external activation unit is a headpiece or is configured to be mounted on a headpiece. The headpiece is configured to be worn on/attached to the head of the subject.

According to some embodiments, the headpiece is a headset, a headband, a head cap, or a hat.

According to some embodiments, the headpiece is further configured to automatically transmit power to the implantable power receiver when the headpiece is positioned on the head of the subject in a predetermined arrangement/position.

According to some embodiments, the external activation unit is further configured to prevent WPT to the power receiver if the headpiece is not positioned on the head of the subject in the predetermined arrangement/position.

According to some embodiments, the external activation unit is communicatively associated with the catheter system.

According to some embodiments, the power transmitter and the implantable power receiver are configured to communicatively associate the external activation unit and the catheter system.

According to some embodiments, the external activation unit includes a user interface allowing a user to operate the external activation unit.

According to some embodiments, the external activation unit further includes a first communication antenna, and the catheter system includes a second communication antenna. The communication antennas are configured to communicatively associate the external activation unit and the catheter system.

According to some embodiments, the implantable controller is configured to prevent the activation of the cleaning unit if the power received by the power receiver is above an upper power threshold.

According to some embodiments, the implantable controller is configured to induce an electrical disconnection between the power receiver and the cleaning unit if the power received by the power receiver exceeds an upper power threshold.

According to some embodiments, the implantable controller is configured to prevent the activation of the cleaning unit if the power received by the implantable power receiver is below a lower power threshold.

According to some embodiments, the implantable controller is configured to prevent activation of the cleaning unit if the received power does not originate from the power transmitter.

According to some embodiments, the external activation unit is configured to transfer power to the implantable power receiver for a predetermined period of time, such as to allow the catheter system to initiate and complete the cleaning session.

According to some embodiments, the implantable controller is further configured to perform an automatic shutdown procedure when the duration of the WPT exceeds an upper time threshold.

According to some embodiments, the external activation unit includes a power source or is configured to be connected to an external power source.

According to some embodiments, the external activation unit includes a battery configured to power the external activation unit (and, in particular, to supply the energy for the WPT).

According to some embodiments, the battery is replaceable and/or rechargeable.

According to some embodiments, the battery may be charged by mounting the external activation unit on a dedicated docking station.

According to some embodiments, the external activation unit includes, or is configured to be connected to, at least one feedback component. The feedback component is configured to output one or more feedback signals indicating one or more of: that the power transmitter is transferring power to the implantable power receiver, that a cleaning session is being effected, and that a cleaning session has terminated.

According to some embodiments, the at least one feedback component includes one or more of: a speaker configured to output one or more audio signals, and a visual component configured to output one or more visual signals.

According to some embodiments, the feedback component is configured to output the one or more feedback signals only when the headpiece is positioned on the head of the patient in the predetermined arrangement/position.

According to some embodiments, the one or more audio signals include music or spoken word(s).

According to some embodiments, the visual component is light source or a display.

According to some embodiments, the processing circuitry is configured to receive a signal indicative of an intracranial pressure and to trigger an alert when the received signal indicates that the intracranial pressure exceeds a predetermined pressure threshold.

According to some embodiments, the processing circuitry is configured to receive a signal indicative of a fluid flow through the catheter system and to trigger an alert when the received signal is indicative of a rate of the fluid flow falling below a predetermined flow rate threshold.

According to some embodiments, the external activation unit is communicatively associated with a mobile communication device.

According to some embodiments, the mobile communication device includes at least one of a smartphone, a smartwatch, a tablet, and a laptop.

According to some embodiments, the external activation unit is further configured to be operated/controlled using software installable on the mobile communication device.

According to some embodiments, the software is configured to allow a user to operate/control the external activation unit, using a user interface of the mobile communication device.

According to some embodiments, the software is configured to trigger a reminder to manually initiate a cleaning session according to a cleaning schedule which is stored in a memory of the mobile communication device and/or which is wirelessly accessible (e.g. stored in a server).

According to some embodiments, the software is configured to automatically initiate cleaning sessions according to a cleaning schedule which is stored in a memory of the mobile communication device and/or which is wirelessly accessible.

According to some embodiments, the software is configured to provide a compliance reward when a cleaning session is completed.

According to some embodiments, the processing circuitry is configured to output to the mobile communication device a signal indicating that the headpiece is properly positioned on the head of subject head.

According to some embodiments, the processing circuitry is configured to output a signal to the mobile communication device when a cleaning session is over. The mobile communication device may be configured to notify the subject to remove the headpiece on receipt of the signal.

According to some embodiments, the processing circuitry is further configured to receive data indicative of occlusion of the catheter system, and to analyze and/or output the data.

According to some embodiments, the body cavity is a brain ventricle, and the data includes intracranial pressure measurement data.

According to some embodiments, the data includes measurement data of fluid flow rate in the catheter.

According to some embodiments, the data is output to the mobile communication device, and the software is configured to process the data to determine whether the catheter is at least partially occluded and/or whether a cleaning session is required.

According to some embodiments, the software is further configured to process the data to determine a degree of occlusion of the catheter system and/or a time for a next cleaning session.

According to some embodiments, the software is configured to determine the time for the next cleaning session using trend analysis, taking into account data received prior to one or more previous cleaning sessions.

According to some embodiments, the body cavity includes a brain ventricle, the fluid includes cerebrospinal fluid, and the external activation unit is associated with a pillow or a mattress.

According to some embodiments, the catheter includes a catheter tube and a catheter tip member fluidly connected to the catheter tube and housing the cleaning unit.

According to some embodiments, a catheter section of the catheter includes one or more apertures fluidly coupling the catheter to the outside thereof. The cleaning unit is configured such as to mechanically prevent, remove, and/or mitigate blockage in at least the catheter section and/or the one or more apertures.

According to some embodiments, the catheter section is or includes the catheter tip member.

According to some embodiments, the cleaning unit includes an elongated shaft including one or more arms configured to project into the one or more apertures and move therein.

According to some embodiments, the cleaning unit is configured to allow vibration thereof. The movement of the one or more arms within the one or more apertures may be induced by the vibration of the cleaning unit.

According to some embodiments, the catheter system further includes a motion generator (e.g. a vibration generator) functionally associated with the implantable controller and configured to induce the motion of the cleaning unit.

According to some embodiments, the motion generator is an electromagnet and the cleaning unit includes, or is mechanically coupled to, a magnet of the electromagnet.

According to an aspect of some embodiments, there is provided a kit including a catheter system, and an external activation unit, as described above.

According to some embodiments, wherein the external activation unit includes a rechargeable battery, the kit may further include a charger to charge the battery.

According to some embodiments, the charger may be a docking station whereon the external activation unit is configured to be mounted for charging.

According to an aspect of some embodiments, there is provided a computer processor configured to execute software instructions configured to control/operate an external activation unit as described above.

According to some embodiments, the computer processor is configured to be installed in a mobile communication device as described above.

According to some embodiments, the software instructions are configured to enable operating/controlling the external activation unit via a user interface of the mobile communication device.

According to an aspect of some embodiments, there is provided a computer-readable storage medium having stored thereon software instructions executable by a computer processor. The software instructions are configured to control/operate an external activation unit as described above.

According to some embodiments, the storage medium is a non-transient memory configured to be installed in a mobile communication device as described above.

According to some embodiments, the software instructions are configured to enable operating/controlling the external activation unit via a user interface of the mobile communication device.

According to an aspect of some embodiments, there is provided a self-cleaning catheter system for draining cerebrospinal fluid (CSF) from the brain of a subject. The catheter system includes an implantable catheter, and an implantable and electrically powered motion actuator. The catheter includes a catheter distal section configured to be implanted in a cavity inside the skull of the subject. The catheter distal section includes (i) one or more apertures, which fluidly couple the catheter to the cavity, and (ii) a cleaning unit configured for motion inside the catheter distal section such as to mechanically prevent, remove, and/or mitigate occlusion therein and/or in at least one of the one or more apertures. The motion actuator is configured to be implanted outside the brain and to be mechanically coupled to the cleaning unit such that by actuating the motion actuator, motion of the cleaning unit is mechanically induced.

According to some embodiments, the cavity includes a brain ventricle.

According to some embodiments, the motion actuator is configured to be implanted outside the skull.

According to some embodiments, the catheter system further includes an elongated extender element connected on a first end thereof to the motion actuator and on a second end thereof to the cleaning unit. The extender element provides the mechanical coupling between the cleaning unit and the motion actuator.

According to some embodiments, the extender element is or includes at least one resilient rod or wire.

According to some embodiments, the motion actuator is an electro-mechanical motor.

According to some embodiments, the motion actuator is a piezoelectric motor.

According to some embodiments, the motion actuator is configured to be ultrasonically powered.

According to some embodiments, the electro-mechanical motor includes an electromagnet.

According to some embodiments, the motion actuator is configured to be implanted in/on the head.

According to some embodiments, the motion actuator is configured to be fully implanted under the skin of the head.

According to some embodiments, the cleaning unit is configured for reciprocal motion, rotational motion, vibrational motion, oscillatory motion, axial motion, radial motion, tilting, and/or any combination thereof.

According to some embodiments, the catheter system further includes a micro-controller) configured to be implanted outside the skull. The controller is functionally associated with the motion actuator.

According to some embodiments, the catheter system further includes an implantable casing, an implantable compartment positioned adjacently to the catheter, and a flexible extension connected on a first end thereof to the casing and on a second end thereof to the compartment, such as to form a Y-shaped arrangement with the catheter. The casing houses the controller, and the compartment houses the motion actuator. The compartment and the casing may both be configured to be implanted outside the skull. Alternatively, the compartment may be configured to be implanted outside the brain (and the casing may be configured to be implanted outside the skull).

According to some embodiments, the motion actuator is electrically connected to the controller by one or more electrical wires, or by a flexible printed circuit board strip, extending through the flexible extension.

According to some embodiments, the catheter system further includes an implantable casing, and a flexible extension connected on a first end thereof to the casing and on a second end thereof to the catheter, such as to form a Y-junction with the catheter. The casing houses the controller and the motion actuator (and may be configured to be implanted outside the skull).

According to some embodiments, a proximal section of the extender element extends through the flexible extension.

According to some embodiments, the extender element is bent such as to conform to an angle defined by the catheter and the flexible extension at the Y-junction. (More specifically, the angle defined by the flexible extension and the distal section of the catheter.) According to some such embodiments, the angle may be obtuse.

According to some embodiments, the catheter includes two lumens: a first lumen, extending along the full length of the catheter and configured for CSF passage therethrough, and, a second lumen, extending along the catheter distal section and connected on a proximal end thereof to the flexible extension. The extender element extends through the second lumen.

According to some embodiments, a proximal end of the catheter is configured to be connected to an implantable valve and/or pump for evacuating fluid from the catheter.

According to some embodiments, the catheter system further includes the valve and/or the pump.

According to some embodiments, the catheter system further includes an implantable power receiver configured to receive power by wireless power transfer (WPT). The power receiver is configured to be implanted outside the skull and to be electrically coupled to the motion actuator and to supply power thereto.

According to some embodiments, the power receiver is functionally associated with the controller and is housed within the casing.

According to some embodiments, the catheter system further includes a power source (e.g. battery) configured to be implanted outside the brain, to be electrically coupled to the motion actuator, and to supply power thereto.

According to some embodiments, the power source is functionally associated with the controller and is housed within the casing.

According to some embodiments, the catheter distal section includes a catheter tip member, which includes at least one of the one or more apertures.

According to some embodiments, the catheter includes a catheter tube fluidly connected to the catheter tip member. The catheter tip member at least partially houses the cleaning unit.

According to some embodiments, the cleaning unit includes an elongated shaft including one or more arms configured to project into the one or more apertures and to move therein. The movement of the one or more arms may prevent at least tissue (and/or other biological material) from entering at least some of the one or more apertures when the catheter tip member is implanted within the body cavity.

According to some embodiments, the cleaning unit is configured to allow vibration thereof. The movement of the one or more arms within the one or more apertures may be induced by the vibration of the cleaning unit.

According to some embodiments, each of one or more electronic components involved in powering the motion is positioned outside the brain.

According to some embodiments, each of the one or more electronic components involved in powering the motion is positioned outside the skull.

According to an aspect of some embodiments, there is provided a kit including a catheter system as described in the description of the previous aspect (describing the catheter system which includes the motion actuator which is configured to be implanted outside the brain and to be mechanically coupled to the cleaning unit of the catheter system, such that by actuating the motion actuator, motion of the cleaning unit is mechanically induced), and a headset configured to be worn on the head of the subject and including a power transmitter configured for WPT to the power receiver.

Aspects of the disclosure, according to some embodiments, pertain to a system configured to detect at least partial occlusion in an implanted catheter (such as in a shunt of a medical implant), and to trigger self-cleaning in response.

Thus, according to an aspect of some embodiments, there is provided a device for at least partially preventing occlusion of an implanted catheter. The device includes at least one processor configured to:
Receive a signal indicative of at least partial occlusion of the catheter implanted within an anatomical body to drain fluid, wherein the catheter includes a plurality of drainage openings therein, susceptible to blockage.
Send an actuation signal for actuating movement of a cleaning element associated with the implanted catheter when at least partial occlusion thereof is detected.

According to some embodiments, the at least one processor is configured to send the actuation signal to an implanted circuit associated with the implanted catheter.

According to some embodiments, the implanted circuit is configured to actuate movement of the cleaning element upon receiving the actuation signal from the at least one processor.

According to some embodiments, the actuation signal includes an alert to a user.

According to some embodiments, the alert advises the user to actuate movement of the cleaning element.

According to some embodiments, the at least one processor is configured to send an additional signal for actuating at least a valve or a pump for evacuating fluid.

According to some embodiments, the device further includes a sensor configured to sense information associated with fluid flow. The at least one processor may be further configured to send the additional signal based on information from the sensor.

According to some embodiments, the valve and the pump are fluidly connected with the catheter.

According to some embodiments, the at least one processor is configured to receive a signal from a sensor incorporated within the catheter.

According to some embodiments, the at least one processor is configured to receive a signal from a sensor incorporated within a valve which is fluidly communicable with the catheter.

According to some embodiments, the implanted catheter is a cerebral shunt for draining cerebrospinal fluid from a ventricle in a brain of subject, and the received signal is indicative of intracranial pressure.

According to some embodiments, the at least one processor is configured to send an actuation signal to the implanted circuit when intracranial pressure falls outside of a predetermined range.

According to some embodiments, the at least one processor is configured to send an actuation signal to actuate a pump for evacuating fluid from the brain ventricle, upon receiving a signal indicating that the intracranial pressure exceeds a predetermined pressure (upper) threshold.

According to some embodiments, the received signal is indicative of fluid flow through the catheter.

According to some embodiments, the at least one processor is configured to send an actuation signal to the implanted circuit when fluid flow through the catheter falls below a predetermined threshold.

According to some embodiments, the at least one processor is further configured to access a treatment schedule and to send an actuation signal to the implanted circuit in accordance with the treatment schedule.

According to some embodiments, the at least one processor is associated with an external activation unit.

According to some embodiments, the external activation unit is wearable.

According to some embodiments, the external wearable activation unit includes a headset configured to be worn on the subject's head.

According to some embodiments, the at least one processor is associated with an implantable activation unit.

According to some embodiments, the at least one processor is configured to receive a control signal from a mobile communication device.

According to some embodiments, the mobile communication device includes at least one of a smartphone, a smartwatch, a tablet, and a laptop.

Aspects of the disclosure, according to some embodiments, pertain to a medical implant including a self-cleaning shunt which may be configured to prevent blockage of the shunt. This may be due, at least in part, to the fact that the shunt may be activated periodically (e.g. daily) by a subject (e.g. patient) wearing an activation headset. The activation headset may allow periodic (e.g. daily) cleaning of the shunt for occlusion prevention thereof.

Thus, according to an aspect of some embodiments, there is provided an apparatus for actuating a self-cleaning shunt implanted in a brain of a subject. The apparatus includes:
A headset configured to be worn on the head of the subject.
An external power source connected to the headset.
An antenna configured for transmitting power from the external power source to an implanted receiver beneath skin on the head of the subject.

The implanted receiver is configured to convey power to the self-cleaning shunt to actuate self-cleaning of the shunt and thereby at least partially prevent occlusion of the shunt.

According to some embodiments, the antenna is configured to automatically transmit power from the external power source to the implanted receiver when the headset is positioned on the head of the subject in a pre-determined arrangement.

According to some embodiments, the antenna is configured to transmit power from the external power source to the implanted receiver only when the headset is positioned on the head of the subject in a pre-determined arrangement.

According to some embodiments, the antenna is configured to transmit power to the implanted receiver such that self-cleaning of the shunt is actuated for a predetermined period.

According to some embodiments, the apparatus further includes at least one speaker connected to the headset and configured to output an audio signal when the antenna transmits power from the external power source to the implanted receiver.

According to some embodiments, the audio signal includes music.

According to some embodiments, the at least one speaker is configured to output the audio signal when self-cleaning of the shunt is actuated.

According to some embodiments, the at least one speaker is configured to output the audio signal only when the headset is positioned on the head of the subject in a pre-determined arrangement.

According to some embodiments, the apparatus further includes a processor configured to control actuation of self-cleaning of the shunt. The processor may be configured to prohibit self-cleaning of the shunt when the implanted receiver receives power from an antenna which is not associated with the headset.

According to some embodiments, the processor is further configured to prohibit self-cleaning of the shunt when the implanted receiver receives a signal which has a magnitude above a pre-determined threshold.

According to some embodiments, the processor is further configured to cause an electrical disconnection when the receiver receives a signal which has a magnitude above a pre-determined threshold.

According to some embodiments, the processor is further configured to perform an automatic shutoff procedure when a time threshold is surpassed.

According to some embodiments, the antenna is further configured to at least one of transmit and receive data from the implanted receiver.

According to some embodiments, the apparatus further includes at least one visual component configured for connection to the headset. The at least one visual component is further configured to output a visual signal when the antenna transmits power from the external power source to the implanted receiver.

According to some embodiments, the visual signal includes light.

According to some embodiments, the at least one visual component includes a light emitting component.

According to some embodiments, the at least one visual component includes a display.

According to some embodiments, the apparatus further includes at least one processor configured to receive a signal indicative of intracranial pressure and to generate an alert when the intracranial pressure falls outside of a predetermined range.

According to some embodiments, the apparatus further includes at least one processor configured to receive a signal indicative of fluid flow through the shunt and to generate an alert when the flow falls below a predetermined threshold.

Aspects of the disclosure, according to some embodiments, pertain to a smartphone application which may execute a treatment protocol for periodical (e.g. daily) clearing of an implanted shunt.

Thus, according to an aspect of some embodiments, there is provided a headset configured to control an implanted self-cleaning shunt, implanted within a head of a subject, via instructions provided by a mobile communications device. The headset includes:
A head band, configured to be worn on the subject's head, and containing an antenna configured to transmit power to a self-cleaning shunt implanted in the subject's head.
A receiver, associated with the band, the receiver being configured to receive from an application running on a mobile communications device, a signal for activating the power transmission to the self-cleaning shunt, wherein the application on the mobile communications device is configured to provide to the subject a reminder to don the band.
At least one processor configured to output to the mobile communications device a signal indicating that the head band is properly positioned on the subject's head and send an actuation signal to actuate the self-cleaning shunt.

According to some embodiments, the processor is further configured to monitor an actuation time of the self-cleaning shunt, and output a signal to the mobile communications device to notify the subject to remove the head band.

According to some embodiments, the at least one processor is further configured to monitor a status of the self-cleaning shunt to determine errors in operation thereof.

According to some embodiments, the at least one processor is further configured to receive data pertaining to operation of the self-cleaning shunt.

According to some embodiments, the at least one processor is further configured to provide an error warning when an operation error of the self-cleaning shunt is detected.

According to some embodiments, the at least one processor is further configured to collect and output data pertaining to at least one dynamic parameter of cerebrospinal fluid.

According to some embodiments, the at least one processor is further configured to collect and output data pertaining to at least partial occlusion of the shunt.

According to some embodiments, the at least one processor is further configured to output music to headphones associated with the headset when the headset is properly placed on the subject's head.

According to some embodiments, the at least one processor is further configured to provide an audible indication when self-cleaning of the shunt is completed.

According to some embodiments, the at least one processor is further configured to provide a visual indication when self-cleaning of the shunt is completed.

According to some embodiments, the application is configured to provide a compliance reward to the subject.

According to some embodiments, the mobile communications device includes at least one of a smartphone, a smartwatch, a tablet, and a laptop.

According to some embodiments, the at least one processor is further configured to receive a signal indicative of intracranial pressure and to generate an alert when the intracranial pressure falls outside of a predetermined range.

According to some embodiments, the at least one processor is further configured to receive a signal indicative of fluid flow through the self-cleaning shunt and to generate an alert when the flow falls below a predetermined threshold.

Aspects of the disclosure, according to some embodiments, pertain to a coil which may be contained within a medical implant to power movement of a cleaning element.

Thus, according to an aspect of some embodiments, there is provided a device for mitigating obstructions in a medical implant. The device includes:
A tubular conduit having a plurality of fluid openings and configured for implantation within an anatomical body for at least one of fluid delivery, fluid drainage, and fluid passage.
A cleaning element located at least partially within the tubular conduit and configured to move within the tubular conduit to mitigate obstruction of the plurality of fluid openings.
A coil associated with the tubular conduit and configured to actuate movement of the cleaning element within the tubular conduit and relative to the plurality of fluid openings.

According to some embodiments, the coil is located within the conduit.

According to some embodiments, the coil is located externally to the conduit.

According to some embodiments, the device further includes a magnet connected to the cleaning element. The magnet is configured to move the cleaning element in response to an electromagnetic field generated by the coil.

According to some embodiments, the coil may be configured to generate an electromagnetic field that causes the magnet to rotate the cleaning element within the tubular conduit.

According to some embodiments, the coil may be configured to generate an electromagnetic field that causes the magnet to move the cleaning element at least one of axially and radially within the tubular conduit.

According to some embodiments, at least a portion of the coil is positioned around at least a portion of the magnet.

According to some embodiments, the coil is positioned around at least a portion of the cleaning element.

According to some embodiments, the fluid openings are positioned within a fluid receiving tip of the tubular conduit. The coil may be positioned in proximity to the fluid receiving tip of the tubular conduit.

According to some embodiments, the coil is embedded within a wall section of the tubular conduit.

According to some embodiments, the cleaning element and at least a portion of the conduit are constructed of titanium.

According to some embodiments, the cleaning element and the conduit are constructed of silicone.

According to some embodiments, the device further includes an antenna configured for transmitting power from an external power source to an implanted receiver. The implanted receiver is configured to convey power to the coil to actuate movement of the cleaning element relative to the plurality of fluid openings.

According to some embodiments, the device further includes an implantable power source which is configured to convey power to the coil to actuate movement of the cleaning element relative to the plurality of fluid openings.

Aspects of the disclosure, according to some embodiments, pertain to a cleaning brush which may float within a medical implant without permanent connection to the medical implant.

Thus, according to an aspect of some embodiments, there is provided a device for mitigating obstructions in a medical implant. The device includes:
A tube having a plurality of openings therein and configured for implantation within an anatomical body for at least one of fluid delivery, fluid drainage, and fluid passage.
A cleaning element configured to be positioned within the tube. The cleaning element includes a plurality of protrusions configured to each extend at least partially into one of the plurality of openings.

The cleaning element is configured to (i) move relative to the plurality of openings for at least partial occlusion prevention thereof and (ii) float within the tube without a fixed connection thereto.

According to some embodiments, the cleaning element includes a central stem. The plurality of protrusions may extend from the stem.

According to some embodiments, the plurality of protrusions are flexible such that the stem and at least a first protrusion are configured to move relative to the tube in an event that a second protrusion becomes immovably fixed to the tube.

According to some embodiments, the cleaning element is configured to abut an edge surface of at least one opening.

According to some embodiments, the cleaning element is configured to sweep opposing portions of opening edge surfaces.

According to some embodiments, the cleaning element and the tube are constructed of silicone.

According to some embodiments, the cleaning element and at least a portion of the tube are constructed of titanium.

According to some embodiments, the cleaning element is magnetically powered.

According to some embodiments, the cleaning element is mechanically powered.

According to some embodiments, the tube further includes an inner surface and an outer surface, such that at least one protrusion is configured to substantially extend through its respective opening from the inner surface of the tube to the outer surface of the tube.

According to some embodiments, the cleaning element further includes a common support from which at least one protrusion extends.

According to some embodiments, the support is located within the tube. The at least one protrusion may be configured to extend from the support and through the opening to at least a plane of the outer surface of the tube.

According to some embodiments, the at least one protrusion is configured to extend radially outward beyond the outer surface of the tube.

According to some embodiments, the support is located outside the tube. The at least one protrusion may be configured to extend from the support and through the opening to at least a plane of the inner surface of the tube.

According to some embodiments, the tube includes an inner surface and an outer surface.

Each opening may have an edge extending between the inner surface of the tube and the outer surface of the tube. The cleaning element and/or the at least one protrusion may be configured for axial oscillation generally in a longitudinal direction of the tube, and for radial oscillation toward and away from the inner surface of the tube.

Aspects of the disclosure, according to some embodiments, pertain to a cleaning element which may have a significantly smaller cross-sectional area than the area of a drainage/delivery opening of a medical implant. As a result, the cleaning element does not impede fluid flow through the opening. A first embodiment may pertain to the relative cross-sectional areas of the cleaning element and opening. A second embodiment may pertain to fluid flow through the opening which is substantially unimpeded by the cleaning element.

Thus, according to an aspect of some embodiments, there is provided a device for mitigating obstructions in a medical implant. The device includes:
A tube having at least one opening therein and configured for implantation within an anatomical body for at least one of fluid delivery, fluid drainage, and fluid passage.
A cleaning protrusion having a portion configured to extend at least partially into the opening and which is configured for movement relative to the opening, wherein the portion of the cleaning protrusion has a cross-sectional area which is less than 75% of an area of the opening.
An actuator configured to cause the cleaning protrusion to move within the opening to mitigate occlusion of the opening.

According to some embodiments, at least a portion of the actuator is substantially secured relative to the cleaning protrusion.

According to some embodiments, the cleaning protrusion is configured to contact an edge of the opening.

According to some embodiments, the device further includes additional cleaning protrusions. Each additional cleaning protrusion may be configured to extend at least partially into a corresponding opening.

According to some embodiments, the cross-sectional area of the portion of the cleaning protrusion is significantly smaller than the area of the opening such that when the portion of the cleaning protrusion is in the opening, fluid flow through the opening is substantially unimpeded.

According to some embodiments, the cross-sectional area of the portion of the cleaning protrusion is less than 50% of the area of the opening.

According to some embodiments, the cleaning protrusion is configured for at least one of axial movement relative to the tube and radial movement relative to the tube.

According to some embodiments, the actuator includes a magnet substantially secured relative to the cleaning protrusion, and a coil configured to produce movement of the magnet.

According to some embodiments, the coil is substantially fixed relative to the tube and the magnet is configured for movement relative to the tube.

According to some embodiments, the tube further includes an inner surface and an outer surface. The cleaning protrusion may be configured to substantially extend through the opening from the inner surface of the tube to the outer surface of the tube.

According to some embodiments, the device further includes a common support (e.g. a central shaft) from which the cleaning protrusion extends.

According to some embodiments, the support is located within the tube and the protrusion is configured to extend from the support and through the opening to at least a plane of the outer surface of the tube.

According to some embodiments, the protrusion is configured to extend radially outward beyond the outer surface of the tube.

According to some embodiments, the support is located outside the tube and the protrusion is configured to extend from the support and through the opening to at least a plane of the inner surface of the tube.

According to some embodiments, the tube includes an inner surface and an outer surface. Each opening has an edge extending between the inner surface of the tube and the outer surface of the tube. The cleaning protrusion may be configured for axial oscillation generally in a longitudinal direction of the tube, and for radial oscillation toward and away from the inner surface of the tube.

According to some embodiments, the device further includes an antenna configured for transmitting power from an external power source to an implanted receiver. The implanted receiver is configured to convey power to the actuator to cause/generate movement of the cleaning protrusion relative to the tube.

According to some embodiments, the device further includes an implantable power source which is configured to convey power to the actuator to affect movement of the cleaning protrusion relative to the tube.

According to an aspect of some embodiments, there is provided a device for mitigating obstructions in a medical implant. The device includes:
A tube having at least one opening therein and configured for implantation within an anatomical body for at least one of fluid delivery, fluid drainage, and fluid passage.
A cleaning protrusion having a portion configured to extend at least partially into the opening and configured for movement relative to the opening, wherein the portion of the cleaning protrusion has a cross-sectional area significantly smaller than an area of the opening such that when the portion of the cleaning protrusion is in the opening, fluid flow through the opening is substantially unimpeded.
An actuator configured to cause the cleaning protrusion to move within the opening to mitigate occlusion of the opening.

According to some embodiments, at least a portion of the actuator is substantially secured relative to the cleaning protrusion.

According to some embodiments, the cross-sectional area of the portion of the cleaning protrusion is less than 75% of the area of the opening.

According to some embodiments, the cross-sectional area of the portion of the cleaning protrusion is less than 50% of the area of the opening.

According to some embodiments, the cleaning protrusion is configured to sweep an edge of the opening.

According to some embodiments, the device further includes additional cleaning protrusions. Each additional cleaning protrusion may be configured to extend at least partially into a corresponding opening.

According to some embodiments, the cleaning protrusion is configured for at least one of axial movement relative to the tube and radial movement relative to the tube.

According to some embodiments, the actuator includes a magnet substantially secured relative to the cleaning protrusion, and a coil configured to produce movement of the magnet.

According to some embodiments, the coil is substantially fixed relative to the tube and wherein the magnet is configured for movement relative to the tube.

According to some embodiments, the tube further includes an inner surface and an outer surface, and the cleaning protrusion is configured to substantially extend through the opening from the inner surface of the tube to the outer surface of the tube.

According to some embodiments, the device further includes a common support from which the cleaning protrusion extends.

According to some embodiments, the support is located within the tube and the protrusion is configured to extend from the support and through the opening to at least a plane of the outer surface of the tube.

According to some embodiments, the protrusion is configured to extend radially outward beyond the outer surface of the tube.

According to some embodiments, the support is located outside the tube and the protrusion is configured to extend from the support and through the opening to at least a plane of the inner surface of the tube.

According to some embodiments, the tube includes an inner surface and an outer surface. Each opening has an edge extending between the inner surface of the tube and the outer surface of the tube. The cleaning protrusion may be configured for axial oscillation generally in a longitudinal direction of the tube, and for radial oscillation toward and away from the inner surface of the tube.

According to some embodiments, the device further includes an antenna configured for transmitting power from an external power source to an implanted receiver. The implanted receiver is configured to convey power to the actuator to cause/generate movement of the cleaning protrusion relative to the tube.

According to some embodiments, the device further includes an implantable power source which is configured to convey power to the actuator to affect movement of the cleaning protrusion relative to the tube.

Aspects of the disclosure, according to some embodiments, pertain to a medical implant which may have an elongated extender connected to a cleaning element for enabling actuation of the cleaning element from a location remote from a tip end of the medical implant.

Thus, according to an aspect of some embodiments, there is provided a device for mitigating obstructions in a medical implant. The device includes:
A fluid receiving tip, having a plurality of fluid openings therein and configured for implantation within an anatomical body for at least one of fluid delivery, fluid drainage, and fluid passage.
A cleaning element configured for movement within the fluid receiving tip to maintain fluid flow through the plurality of fluid openings.
A lumen extending from the fluid receiving tip. The lumen has a distal portion fluidly communicable with the fluid receiving tip, and a proximal portion spaced from the distal portion.
An actuator positioned at a location spaced from the distal portion of the lumen.
An extender, passing through the lumen and interconnecting the actuator and the cleaning element. The extender is configured to mechanically transfer to the cleaning element, through the lumen, movement generated by the actuator.

According to some embodiments, the cleaning element is configured for axial oscillatory movement within the fluid receiving tip. The actuator is configured to generate axial oscillatory movement. The extender is configured to transfer the axial oscillatory movement of the actuator to the cleaning element.

According to some embodiments, the cleaning element is configured for rotational movement within the fluid receiving tip. The actuator is configured to generate rotational movement. The extender is configured to transfer the rotational movement of the actuator to the cleaning element.

According to some embodiments, the device further includes an antenna configured for transmitting power from an external power source to an implanted receiver. The implanted receiver is configured to convey power to the actuator to affect movement generation by the actuator.

According to some embodiments, the actuator is positioned in proximity to the implanted receiver (e.g. closer to the receiver than to the cleaning element).

According to some embodiments, the device further includes an implantable power source which is configured to convey power to the actuator to affect movement generation by the actuator.

According to some embodiments, the fluid receiving tip and the cleaning element are constructed of silicone.

According to some embodiments, the actuator is a piezoelectric actuator.

According to some embodiments, the actuator is ultrasonically activated.

According to some embodiments, the extender is configured to hydraulically transfer the movement generated by the actuator.

According to some embodiments, the extender is configured to magnetically transfer the movement generated by the actuator.

Certain embodiments of the present disclosure may include some, all, or none of the above advantages. One or more other technical advantages may be readily apparent to those skilled in the art from the figures, descriptions, and claims included herein.

Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging”, “concluding”, “establishing”, or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A schematically depicts a prior art cerebral shunt for draining cerebrospinal fluid from a ventricle in a brain of subject;

FIG. 1B schematically depicts a prior art ventricular catheter assembly of the cerebral shunt of FIG. 1A;

FIG. 2 is a block diagram of a catheter kit including an implantable, self-cleaning catheter system and an external activation unit functionally associated with the catheter system and configured to power a cleaning session of the catheter system, according to some embodiments;

FIG. 3 is a block diagram of the catheter kit of FIG. 2 and a mobile communication device communicatively associated with the external activation unit, according to some embodiments;

FIG. 4 is a block diagram of an implantable, self-cleaning catheter system with occlusion detection capabilities, and an external controller functionally associated with the catheter system, according to some embodiments;

FIG. 5 is a schematic, perspective view of an implantable catheter system, which is a specific embodiment of the catheter system of FIG. 2, the catheter system includes a catheter, a casing, and a flexible extension, according to some embodiments;

FIG. 6 is a schematic, perspective view of a catheter tip member and a tube distal section of the catheter of FIG. 5, according to some embodiments;

FIG. 7 is a schematic, perspective view of a cleaning unit and a vibration generator of the catheter of FIG. 5, according to some embodiments;

FIGS. 8A-8C are schematic, cutaway views of the catheter tip member of FIG. 6 illustrating motion of the cleaning unit of FIG. 7 in the catheter tip member during a cleaning session, according to some embodiments;

FIG. 9 is a schematic, cross-sectional view of a tip member distal section of the catheter tip member of FIG. 6, the cross-section is taken along a plane perpendicular to a longitudinal axis of the catheter tip member, according to some embodiments;

FIG. 10 is a schematic, perspective view of a catheter assembly for draining cerebrospinal fluids from the brain, the catheter assembly including the catheter system of FIG. 5, according to some embodiments;

FIG. 11 schematically depicts a subject implanted with the catheter assembly of FIG. 10 and wearing a headset configured for powering the catheter system and initiating a cleaning session, according to some embodiments;

FIGS. 12A and 12B schematically depicts the catheter assembly of FIG. 10 and the headset of FIG. 11, the headset is shown in two configurations between which the headset is controllably switchable, according to some embodiments;

FIG. 13 schematically depicts the catheter assembly of FIG. 10 and the headset of FIG. 11 positioned relative to one another such to allow powering the catheter assembly by the headset, according to some embodiments;

FIG. 14 schematically depicts the subject of FIG. 11 when wearing the headset of FIG. 11 with an indicator light on a user interface of the headset switched on, according to some embodiments;

FIG. 15 is a schematic, perspective view of an implantable catheter system, which is a specific embodiment of the catheter system of FIG. 2, the catheter system includes a catheter, a casing, and a flexible extension, according to some embodiments;

FIG. 16 is a schematic, perspective, partial view of a catheter tip member of the catheter of FIG. 15, the catheter tip member housing a cleaning unit, according to some embodiments;

FIG. 17 is a schematic, perspective, partial view of the casing of FIG. 15, the casing housing a motion actuator, according to some embodiments;

FIG. 18 is a schematic, perspective view of an implantable catheter system, which is a specific embodiment of the catheter system of FIG. 2, the catheter system includes a catheter, a casing, a flexible extension, and a compartment, according to some embodiments; and

FIG. 19 is a schematic, perspective view of the compartment of FIG. 18, the compartment housing a motion actuator, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application the expression “at least one of A and B”, (e.g. wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

In the description and claims of the application, the words “include” and “have”, and forms thereof, are not limited to members in a list with which the words may be associated.

In figures depicting block diagrams/flowcharts, optional elements/steps may be written within a box delineated by a dashed line.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol ⊙ may be used to represent an axis pointing “out of the page”, while the symbol ⊗ may be used to represent an axis pointing “into the page”.

As used herein, according to some embodiments, a “proximal” end/section/portion/tip of an element/component/device may refer to a part of the element/component/device that is closer to a surgeon or a medical practitioner (e.g. during implantation of the device) as compared to at least one other part of the element/component/device. Similarly, according to some embodiments, a “distal” end/section/portion/tip of an element/component/device may refer to a part of the element/component/device that is further from a surgeon or a medical practitioner (e.g. during implantation of the device) as compared to at least one other part of the element/component/device. According to some embodiments, a “distal” end/section/portion/tip of an element/component/device may refer to a part of the element/component/device that is closer to a diagnosis or treatment site in the body of a subject as compared to at least one other part of the element/component/device.

As used herein, according to some embodiments, the term “implantable” with reference to an object (e.g. medical device or component/element), may refer to (i) an object which is configured to be fully implanted (e.g. a pacemaker) in the sense that when implanted no part of the object is outside the body or exposed on the skin, as well as to (ii) an object which is configured to be partially implanted (e.g. a feeding tube) in the sense that when implanted a part of the object is outside the body or exposed on the skin. According to some embodiments, an element may be said to be “implantable” when housed, or included, in another element which is implantable in the sense defined above.

As used herein, according to some embodiments, the term “fluid passage” is used in a broad sense to cover also one or more of fluid drainage and fluid delivery (supply).

As used herein, according to some embodiments, the term “occlusion” with reference to a conduit/tube/hole/aperture—or any other kind of fluid passage means (in particular, a catheter system, such as the catheter systems disclosed herein)—encompasses both full “blockage” and partial blockage of the conduit/tube/hole/aperture. Similarly, the term blockage may refer both to full blockage and partial blockage.

As used herein, according to some embodiments, the terms “control circuitry” and “processing circuitry” may be used interchangeably.

As used herein, according to some embodiments, the term “communicatively associated”, and terms similar thereto, with reference to two components, may refer both to the case wherein two-way communication between the two components is possible (e.g. when each of the two components is a transceiver or a transmitter-receiver), as well as to the case wherein one of the components is configured to only send signals or only to receive signals (e.g. when one of the components is transmitter and the other component is a receiver or a transceiver).

As used herein, according to some embodiments, the term “electrically associated by wire” and the like, with reference to two electrical components, cover both the case wherein the two components are electrically connected/coupled by a “standard” electrical wire (e.g. a co-axial wire), as well as other cases, such as when the two components are electrically connected/coupled via copper/gold tracks on a printed-circuit board (PCB) or a flexible PCB strip.

As used herein, according to some embodiments, the terms “external controller” and “mobile communication device” may be used interchangeably.

As used herein, according to some embodiments, the term “state of occlusion” refers to the degree of occlusion of a conduit/tube/hole/apertures or any other means for fluid passage. The degree of occlusion may range from no blockage to full blockage.

As used herein, according to some embodiments, the term “control circuitry” refers to electronic circuitry configured to control functions/operations of electronic components/devices. In particular, a control circuitry may include one or more processors, one or more (transient and non-transient) memory components, and an internal clock.

As used herein, according to some embodiments, the term “kit” refers to a plurality of devices/tools which may be used for a (common) specific purpose. According to some embodiments, the terms “kit” and “system” may be used interchangeably.

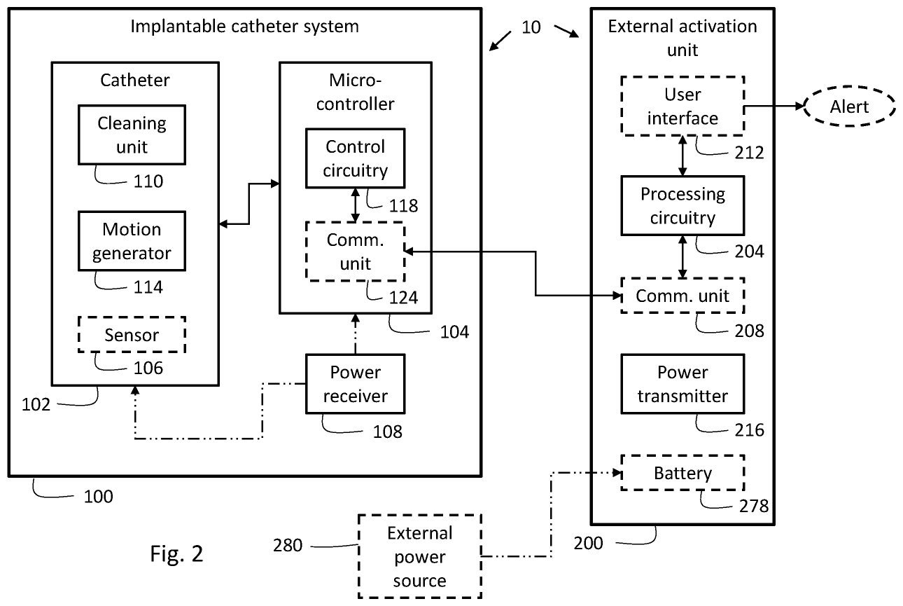

According to an aspect of some embodiments, there is provided an external activation unit for an implantable, self-cleaning catheter system for fluid passage. The activation unit is configured to initiate a cleaning (i.e. self-cleaning) session in the catheter system, as explained below. The activation unit is said to be “external” in the sense of not being implantable and not being part of the catheter system. According to some embodiments, the external activation unit may be provided together with the catheter system as part of a kit, as depicted in FIG. 2.

FIG. 2 is a block diagram of a catheter kit 10 including a self-cleaning, implantable catheter system 100 configured for fluid passage, and an external activation unit 200 functionally associated therewith, according to some embodiments. Catheter system 100 includes an implantable catheter 102 (or more generally, an implantable shunt and/or delivery port), an implantable micro-controller 104, and an implantable power receiver 108 (e.g. an inductive antenna).

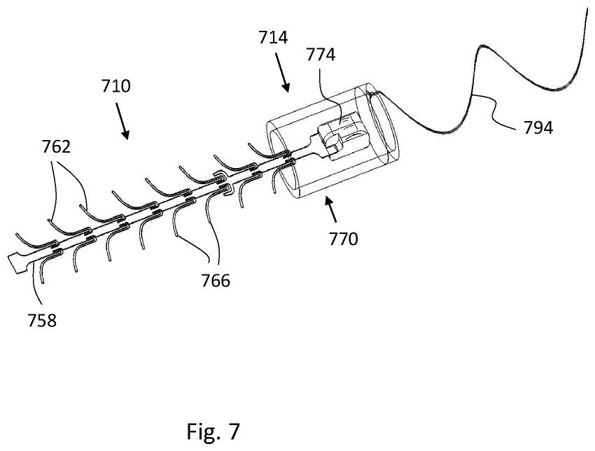

Catheter 102 is configured to be implanted in a body cavity and/or lumen. According to some embodiments, catheter 102 is configured to drain fluids (body fluids) from the body cavity/lumen, and/or to deliver fluids (e.g. medication) to the body cavity/lumen. Catheter 102 includes a cleaning unit 110 housed therein. Cleaning unit 110 is configured for motion (e.g. reciprocal and/or rotational motion, vibration) within catheter 102, such as to clean at least a section of catheter 102. More specifically, cleaning unit 110 is configured to mechanically prevent, remove, or at least mitigate, blockage(s) in catheter 102, such as to maintain fluid flow through catheter 102 (or the possibility for fluid flow therethrough), as elaborated on below. According to some embodiments, and as depicted in FIG. 2, catheter 102 further includes a motion generator 114 configured to generate cleaning unit 110 motion, as explained below. According to some other embodiments, motion generator 114 is not included in catheter 102 and may be configured to be implanted separately therefrom. According to some embodiments, motion generator 114 is mechanically associated with cleaning unit 110. According to some embodiments, motion generator 114, or a part thereof, forms part of cleaning unit 110, or is attached thereto. For example, in embodiments wherein motion generator 114 is an electromagnet, the magnet of the electromagnet may form a part of, or be attached to, cleaning unit 110, as depicted, for example, in FIG. 7 and explained in the description thereof. According to some embodiments, and as elaborated on below, motion generator 114 is a piezoelectric motor.

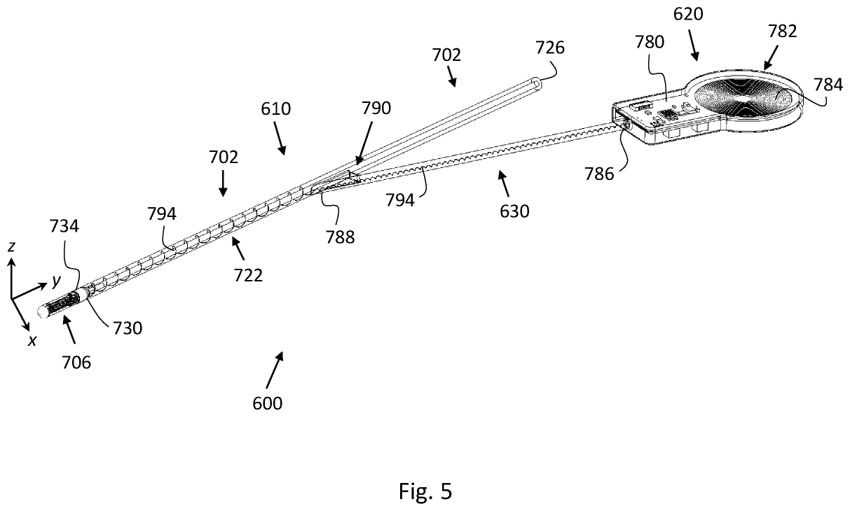

Power receiver 108 is configured to receive energy by wireless power transfer (WPT) and to power cleaning unit 110. According to some embodiments, power receiver 108 also powers micro-controller 104. According to some embodiments, micro-controller 104 and power receiver 108 are both housed in a common casing (such as the casing depicted in FIG. 5) that is implantable.

Micro-controller 104 is functionally associated with power receiver 108 and cleaning unit 110. Micro-controller 104 includes a control circuitry 118 (e.g. electronic components, processor(s)). Control circuitry 118 may be configured to command power receiver 108 and cleaning unit 110, e.g. to activate/deactivate cleaning unit 110 and/or to receive a signal from power receiver 108 signifying that power receiver 108 is being powered.

External activation unit 200 includes a processing circuitry 204 (e.g. a computer processor and non-transient memory), a power transmitter 216, and, optionally, a user interface 212, which may allow a user (e.g. the subject, a caretaker, medical personnel) to operate external activation unit 200. Power transmitter 216 and user interface 212 are both functionally associated with processing circuitry 204. In particular, processing circuitry 204 may be configured to command power transmitter 216 to start/stop transmitting power, as elaborated on below.

External activation unit 200 is configured for WPT to catheter system 100. More specifically, external activation unit 200 and catheter system 100 are configured such that, in operation, power transmitter 216 transmits energy to power receiver 108. According to some embodiments, wherein the WPT is based on inductive coupling, each of power receiver 108 and power transmitter 216 may be a coil of conducting wire. According to some embodiments, wherein the WPT is based on capacitive coupling, each of power receiver 108 and power transmitter 216 may be a metal electrode.

According to some embodiments, micro-controller 104 further includes a communication unit 124 (e.g. a Bluetooth or RF antenna) communicatively associated (e.g. by wire) with control circuitry 118. According to some embodiments, external activation unit 200 includes a communication unit 208 (e.g. a Bluetooth or RF antenna) communicatively associated (e.g. by wire) with processing circuitry 204. In such embodiments, catheter system 100 and external activation unit 200 (i.e. control circuitry 118 and processing circuitry 204, respectively) may be communicatively associated via communication units 124 and 208.

According to some embodiments, communication unit 124 is a receiver and communication unit 208 is a transmitter. According to some embodiments, each of communication unit 124 and communication unit 208 is a transceiver or a transmission-receiver.

According to some embodiments, not depicted in FIG. 2, micro-controller 104 does not include communication unit 124: Instead, power receiver 108 includes communication unit 124. That is, power receiver 108 is further used to communicatively associate catheter system 100 with external activation unit 200. Similarly, according to some such embodiments, power transmitter 216 may include communication unit 208. For example, in embodiments wherein each of power receiver 108 and power transmitter 216 includes a coil of conducting wire and is configured for WPT by inductive coupling there between, power receiver 108 may further be used to communicatively associate control circuitry 118 and processing circuitry 204. In particular, power receiver 108 may further be used to convey instructions from processing circuitry 204 to control circuitry 118, and/or to send data from control circuitry 118 to processing circuitry 204. According to some such embodiments, power transmitter 216 is similarly used to communicatively associate control circuitry 118 and processing circuitry 204

According to some embodiments, catheter system 100 may be configured to prevent activation of cleaning unit 110 if power receiver 108 receives power from any power source other than external activation unit 200, e.g. when power receiver 108 is exposed to a magnetic field which is not generated by power transmitter 216. To verify that the power received is actually from power transmitter 216, communication antenna 208/power transmitter 216 may be configured to send an authentication signal, as known in the art, e.g. a Bluetooth authentication signal when communication antennas 124 and 208 are Bluetooth antennas. Control circuitry 118 may be configured to “forget” external activation unit 200 once a cleaning session has been completed, or after a pre-determined time interval has passed, in the sense of requiring re-authentication before each new cleaning session. According to some embodiments, catheter system 100 includes an electrical switch (not shown) which is configured to electrically couple/decouple power receiver 108 and cleaning unit 110. In such embodiments, micro-controller 104 may be configured to close the switch (thereby electrically coupling cleaning unit 110 to power receiver 108) when power received by power receiver 108 is determined to originate from power transmitter 216.

According to some embodiments, micro-controller 104 may be configured to prevent activation of cleaning unit 110 if power receiver 108 receives power above a pre-determined threshold. According to some embodiments, micro-controller 104 may be configured to induce an electrical disconnection between power receiver 108 and cleaning unit 110 when power received by power receiver 108 is above the predetermined threshold. For example, when catheter system 100 includes an electrical switch, as described above, micro-controller 104 may be configured to not close the electrical switch when the power received is above the predetermined threshold. According to some embodiments, the predetermined threshold may be selected such as to ensure that during a cleaning session the temperature of power receiver 108 does not increase by more than, for example, about 5° C. (Celsius), about 3° C., about 2° C., or even about 1° C.

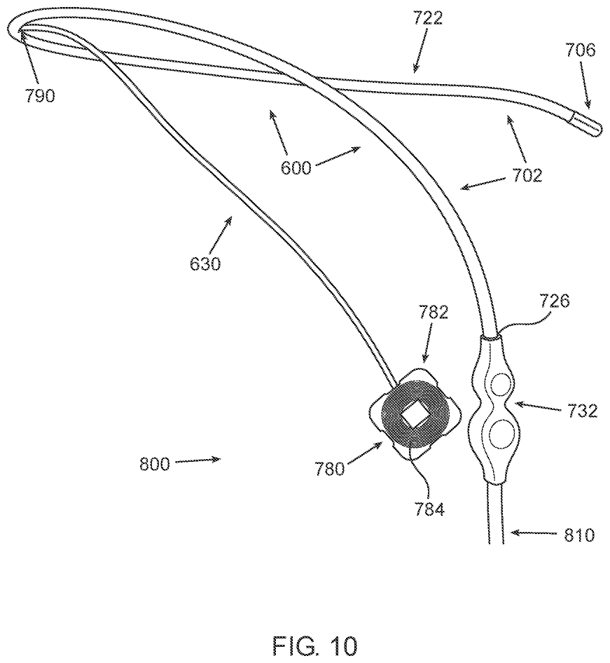

According to some embodiments, external activation unit 200 is wearable. According to some embodiments, wherein catheter system 100 is a ventricular catheter system for draining CSF fluid from a brain ventricle, external activation unit 200 may be a headpiece configured to be worn by the subject. According to some embodiments, external activation unit 200 is a headset, essentially as depicted in FIG. 10, and as elaborated on below. According to some embodiments, external activation unit 200 is a hat, a headband, or a head cap.

According to some embodiments, wherein external activation unit 200 is wearable, external activation unit 200 is configured to transfer power to catheter system 100 only upon manual activation by the user/caregiver, e.g. using user interface 212 or dedicated software (e.g. app) on a mobile communication device, as described below.

According to some embodiments, wherein external activation unit 200 is wearable, external activation unit 200 is configured to automatically transfer power to catheter system 100 when worn (thereby allowing automatic initiation of a cleaning session when external activation unit 200 is put on). According to some embodiments, external activation unit 200 is configured to automatically transfer power to catheter system 100, upon being worn, only after a predetermined amount of time has passed since the last cleaning session (initiation and/or completion), e.g. 24 hours.

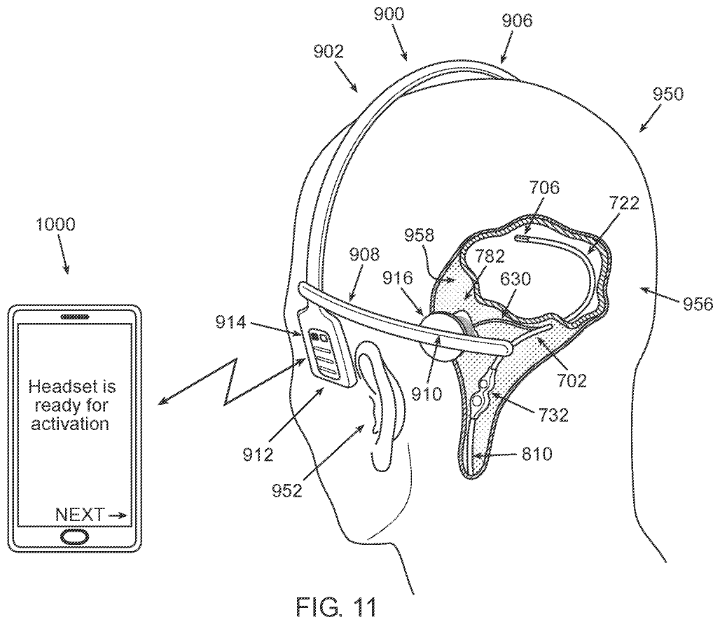

According to some embodiments, wherein external activation unit 200 is wearable, external activation unit 200 is configured to prevent power transfer therefrom if not worn in a predetermined arrangement/position. For example, according to some embodiments wherein external activation unit is a headset, external activation unit 200 may be configured to prevent power transfer therefrom if not positioned on the head of the subject such that power transmitter 216 is adjacent to power receiver 108 (which is implanted beneath the skin outside the skull), essentially as depicted in FIG. 11. According to some such embodiments, external activation unit 200 is configured to automatically transfer power to catheter system 100 when worn in the predetermined arrangement/position.

It is noted that external activation unit 200 may correspond to, or be included in, other articles of clothing according to the body part in which catheter system 100 is configured to be implanted. For example, when catheter system 100 is configured to be implanted in the stomach or the chest, external activation unit 200 may be included in a shirt, an undershirt, or a jacket of a pajamas set.

According to some alternative embodiments, wherein catheter system 100 is a ventricular catheter system for draining CSF fluid from a brain ventricle, external activation unit 200 may be, for example, a pillow or a mattress, or may be configured to be installed in/on a pillow or a mattress. According to some such embodiments, external activation unit 200 may be configured to transmit power to catheter system 100 only when the subject manually activates external activation unit 200. Alternatively, external activation unit 200 may be configured to automatically transmit power to catheter system 100 when the subject places his head on the pillow or the mattress, or when the subject places his head on the pillow or the mattress at a certain arrangement(s) and/or at a certain orientation(s)/positioning(s) relative to the pillow or the mattress.

According to some embodiments, user interface 212 includes a feedback component(s) configured to output one or more feedback signals indicating that the power transmitter is transferring power to the power receiver, that a cleaning session is being effected, and/or that a cleaning session has terminated. According to some embodiments, the feedback component(s) is, or includes, an audio component(s) (e.g. a speaker). In such embodiments, processing circuitry 204 may be configured to switch on the audio component—so that the audio component produces sound—when external activation unit 200 is powering catheter system 100. The sound may be, for example, music or spoken words. The audio component may be configured to produce sound as long as external activation unit 200 is powering catheter system 100, i.e. throughout the entire cleaning session, either continuously or intermittently, or it may be configured to produce sound only when the WPT begins and/or ends in order to indicate the commencement and/or termination of the cleaning session. According to some embodiments, wherein external activation unit 200 is wearable, a second and different sound may be used to signal that that external activation unit is improperly worn (e.g. is not worn in the predetermined arrangement/position). Additionally or alternatively, according to some embodiments, the feedback component(s) is, or includes, a visual component(s) (e.g. an LED light bulb and/or a display), which may be used to the same end, e.g. to produce a visual signal (e.g. a lighting or flickering of the LED light bulb, an image and/or text shown on the display) signifying that external activation unit 200 is powering catheter system 100 and/or that powering of catheter system 100 by external activation unit 200 has started and/or has ended.

According to some embodiments, processing circuitry 204 may have stored in a memory a treatment schedule (e.g. predetermined timing of cleaning sessions, time intervals between consecutive cleaning sessions, the duration of the cleaning sessions). According to some such embodiments, processing circuitry 204 may be configured to command user interface 212 to notify the subject/caretaker that a cleaning session is scheduled. According to some such embodiments, processing circuitry 204 may be configured to automatically launch a cleaning session if external activation unit 200 is properly positioned, such as to allow powering catheter system 100, and a cleaning session is scheduled. According to other such embodiments, launching of the scheduled cleaning session requires manual activation of external activation unit 200, and, thus, processing circuitry 204 may be further configured to command user interface 212 to prompt the subject/caretaker to activate external activation unit 200 once it is properly positioned relative to the subject.

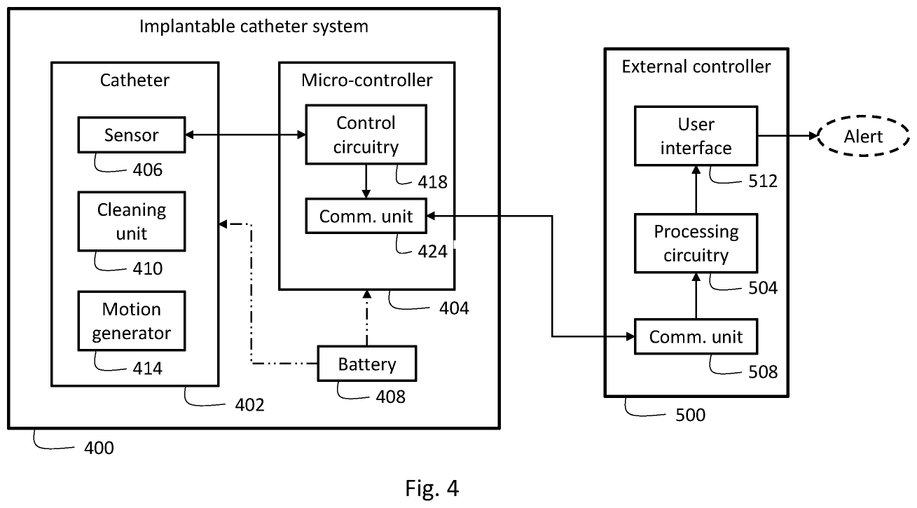

According to some embodiments, not shown in FIG. 2, catheter system 100 further includes at least one implantable sensor 106—which may be essentially similar to the sensor of the catheter system depicted in FIG. 4—and which may be configured to monitor one or more parameters indicative of occlusion in catheter 102 and/or a developing medical condition (e.g. significant accumulation of excess fluid in the body cavity) and/or malfunction of cleaning unit 110. In such embodiments, user interface 212 may further be used to produce a signal or generate an alert when sensor 106 readings indicate that the value(s) of the parameter(s) has fallen outside a predetermined range and/or (sudden) change in a measured value (e.g. sharp increase or decrease). For example, in embodiments wherein catheter system 100 is configured to be implanted in a brain ventricle (and external activation unit 200 is a head piece), sensor 106 may be configured to monitor intracranial pressure with the alarm being generated when sensor 106 readings indicate that the intracranial pressure exceeds a predetermined pressure (upper) threshold or drops below a predetermined pressure (lower) threshold, and/or when sensor 106 readings indicate that there was a rapid increase/decrease in the intracranial pressure. Or, for example, sensor 106 may be configured to monitor fluid flow rate within the catheter with the alarm being generated when the sensor readings indicate that the flow rate has dropped below a predetermined flow rate (lower) threshold and/or a rapid drop of the flow rate. According to some embodiments, the alarm may further be generated when the sensor readings indicate that the flow rate has exceeded a predetermined flow rate (upper) threshold and/or a rapid increase in the flow rate (e.g. due to a malfunction of the fluid-evacuating valve or a rupture in the catheter tube). The produced signal/generated alarm may be aural (generated by the audio component(s)) and/or visual (generated by the visual component(s)). The processing of sensor 106 readings may be carried out by control circuitry 118 and/or by processing circuitry 204. The produced signal may be used to notify the subject or a caretaker thereof (e.g. when the subject is an infant) that a cleaning session is required, while the alarm may be used to notify the subject or a caretaker thereof that medical intervention is required. According to some embodiments, and as depicted in FIG. 2, sensor 106 may be positioned in/on catheter 102. According to some other embodiments, sensor 106 is not included in catheter 102 and may be configured to be implanted separately therefrom.

According to some embodiments, sensor 106 may be configured to measure each time a cleaning session is activated.

The solid lines extending between components in FIGS. 2-4 serve to indicate e.g. information flow and/or instructions, while the dashed-dotted lines (in FIGS. 2 and 4) serve to indicate e.g. power transfer from one component to another.

According to some embodiments, and as depicted in FIG. 2, external activation unit 200 may be powered by battery 278, which is included therein. According to some embodiments, the battery may be rechargeable (e.g. using an electrical/USB port) and/or replaceable. According to some embodiments, external activation unit 200 may be connected to an external power source 280 for charging battery 278. According to some such embodiments, external activation unit 200 may include an electrical port, or an electrical cable with a plug, which is configured to be connected to external power source 280. According to some such embodiments, external activation unit 200 may be configured to be charged using a dedicated charger and/or docking station. According to some such embodiments, processing circuitry 204 is configured not to allow WPT to catheter system 100 when external activation unit 200 is connected to external power source 280. According to some such embodiments, wherein external activation unit 200 is wearable, external activation unit 200 is configured not to allow the wearing thereof when being charged, i.e. external activation unit 200 cannot be worn when connected to external power source 280, such as by requiring external activation unit 200 to be positioned on a docking station for charging, or by requiring an electrical cable to be connected to a recharging port (e.g. USB port) positioned on an inner portion of the wearable external activation unit 200, i.e. a portion facing the subject's body when worn. According to some embodiments, in particular, in embodiments wherein external activation unit 200 does not include battery 278, external activation unit 200 may be configured to be powered directly from an external power source, such as an external battery or even from the electrical system of the home. According to some such embodiments, external activation unit 200 may include a plug configured to be plugged into an electrical outlet (e.g. wall socket).

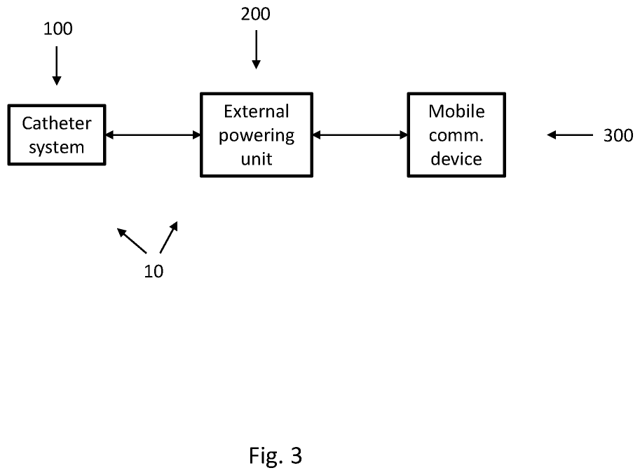

Making reference to FIG. 3, according to some embodiments, external activation unit 200 may be operated (i.e. may be controlled) using a mobile communication device 300 (e.g. a smartphone, a smartwatch, a tablet, a laptop) with which external activation unit 200 is communicatively associated (e.g. via communication unit 208). Mobile communication device 300 may have installed thereon software (e.g. an app) configured to allow a user to operate external activation unit 200, and thereby to operate catheter system 100. In particular, the user may use mobile communication device 300 to have catheter system 100 launch a cleaning session. Further, according to some embodiments, a treatment schedule (cleaning session timetable) may be entered into the app, so that the app may remind the user (e.g. the subject or a caretaker thereof) that a cleaning session will soon be due/is due/is overdue. The reminder may ask the subject to position external activation unit 200 such as to allow powering catheter system 100 (e.g. to don external activation unit 200 when external activation unit is wearable). Further, external activation unit 200 may be configured to communicate to mobile communication device 300 whether external activation unit 200 is ready to power catheter system 100 (e.g. whether external activation unit 200 is properly positioned/worn), and the app may be configured to inform the subject whether a cleaning session may be launched or whether external activation unit 200 has to be e.g. repositioned, connected to external power source 280, etc. According to some such embodiments, mobile communication device 300 may be configured to independently instruct external activation unit 200 to launch a cleaning session when a cleaning session is due, if external activation unit 200 is positioned such as to allow powering catheter system 100 or once the subject has so positioned external activation unit 200.

According to some embodiments, external activation unit 200 may be configured to report to mobile communication device 300 when a cleaning session has ended, and the app may be configured to so inform the user (e.g. so that the subject may remove/take off external activation unit 200).

According to some embodiments, the app may be configured to receive data indicative of occlusion of the catheter system (e.g. sensor 106 data) and, optionally, to process the data to determine a degree of occlusion of the catheter system. In some embodiments, wherein the activation of the cleaning unit is based on the determined degree of occlusion, in addition to, or in place of, periodic cleaning sessions, the app may be further configured to determine a recommended time for a next cleaning session. According to some embodiments, the time for the next cleaning session is determined using trend analysis, taking into account data received prior to one or more previous cleaning sessions. Thus, according to some embodiments, the app may be configured to independently construct the cleaning session schedule. According to some embodiments, the app may be configured to be installable also on non-mobile computational devices, such as a desktop computer (e.g. of a physician of the subject).

More generally, any information listed above in the description of external activation unit 200, which is relayed/communicated to the subject/caretaker using user interface 212, according to some embodiments, may be relayed/communicated instead using mobile communication device 300. In particular, in embodiments wherein user interface 212 includes limited features or wherein external activation unit 200 does not include user interface 212, some or all of the information may be relayed instead using mobile communication device 300. Similarly, according to some embodiments, some or all processing of data (e.g. sensor 106 data) listed above in the description of catheter system 100 and external activation unit 200 and performed in either one of or both of catheter system 100 and external activation unit 200, may be performed instead by mobile communication device 300 (with external activation unit 200 being configured to send the data thereto).

According to some embodiments, the app may be configured to award a compliance reward each time a cleaning session (or a predetermined number thereof) is completed. The compliance reward may be in the form of music and/or may be graphical or even animated (shown on the display of mobile communication device 300). According to some embodiments, the app may include a game with the compliance reward unlocking extra/hidden features of the game.

According to some embodiments, mobile communication device 300 may be configured to share with other computational devices (e.g. other mobile communication devices, as well as non-mobile computational devices, such as a desktop computer at the physician's office, etc.) or a server, information received from external activation unit 200 and/or inform the other computational devices when a next cleaning session is scheduled, when a cleaning session has been concluded, and/or when occlusion is detected. Thus, for example, mobile communcation device 300 may be a mobile communication device of the subject, and the other computational devices may belong e.g. to a caretaker of the subject and/or family members thereof (e.g. smartphones and/or desktop computers of the caretaker(s)/family members). According to some embodiments, a plurality of mobile communication devices, such as mobile communication device 300, may have installed thereon the app described above, and accordingly may be configured to operate external activation unit 200. The plurality of mobile communication devices may belong to the subject, a caretaker(s) thereof, and/or family members thereof, etc. According to some embodiments, some or all of the information, which mobile communication device 300 shares with other computational devices, is encrypted.

According to an aspect of some embodiments, there is provided an implantable, self-cleaning catheter system 400 for fluid passage with occlusion monitoring capabilities. Catheter system 400 includes an implantable catheter 402, an implantable micro-controller 404, and at least one implantable sensor 406. Sensor 406 may be configured to measure/monitor at least one parameter indicative of occlusion in catheter 402, as elaborated on below. Catheter system 400 may be communicatively associated with an external controller 500, such as a mobile communication device, which is configured to allow a user to operate catheter system 400, in particular, to instruct catheter system 400 to initiate a cleaning session. External controller 500 may further be configured to notify the subject (or a caretaker thereof) when a cleaning session is due/required, as elaborated on below. External controller 500 may be similar to mobile communication device 300 but differs therefrom in being configured to directly communicate and operate catheter system 400, whereas mobile communication device 300, according to some embodiments, “interacts” with catheter system 100 via external activation unit 200.

According to some embodiments, and as depicted in FIG. 4, catheter system 400 further includes a battery 408 for powering components thereof. Non-limiting examples of suitable batteries include implantable batteries similar to those used in pacemakers, as well as implantable batteries which are rechargeable by WPT. Catheter system 400 is similar to catheter system 100 but differs therefrom at least in being powered by battery 408 instead of by WPT (though, according to some embodiments, wherein the battery is implantable, the battery may be rechargeable by WPT). According to some embodiments, micro-controller 404 and battery 408 are both housed in a common casing (not shown) which is implantable.

According to some embodiments, not depicted in FIG. 4, catheter system 400 may be configured to be powered by WPT. In such embodiments, catheter system 400 may include a power receiver, similar to power receiver 108.

Making reference again to FIG. 4, according to some embodiments, catheter 402 includes a cleaning unit 410 and a motion generator 414, essentially similar to cleaning unit 110 and motion generator 114, and a wireless communication unit 424 (e.g. a Bluetooth or RF antenna) configured to communicatively associate micro-controller 404 with external controller 500. According to some other embodiments, motion generator 414 is not included in catheter 402 and may be configured to be implanted separately therefrom. Micro-controller 404 is communicatively associated with both cleaning unit 410 and sensor 406. Micro-controller 404 includes a control circuitry 418 (e.g. processor and memory components) configured to command cleaning unit 410 and sensor 406, e.g. to activate/deactivate cleaning unit 410 and/or sensor 406. Sensor 406 is configured to send sensor readings (measurement/monitoring data) thereof to micro-controller 404.

According to some embodiments, external controller 500 includes a processing circuitry 504, a communication unit 508, and a user interface 512. Communication unit 508 and user interface 512 are functionally associated with processing circuitry 504. Communication unit 508 is configured to communicatively associate processing circuitry 504 with micro-controller 404 such as to allow a user to operate catheter system 400 using external controller 500. User interface 512 may include a display (e.g. a touch screen) and/or audio components (e.g. a speaker), knobs/buttons, and so on to allow a user to operate external controller 500, as well as to allow external controller 500 to communicate information, e.g. from catheter system 400, to the user (e.g. to notify the user that a cleaning session is due/required).

According to some embodiments, wherein external controller 500 is a mobile communication device (e.g. a smartphone), external controller 500 may have custom software (e.g. an app) installed thereon. The software may be configured to allow a user to operate catheter system 400. In some embodiments, the software may further be configured to allow a user to manage the data received from catheter system 400, e.g. sensor 406 readings. For example, the software may be configured to perform trend analysis, generate graphs, etc.

It is noted that occlusion in catheter 402 may lead to an increase in pressure inside catheter 402, when catheter system 400 is configured to deliver fluid/s into a body cavity/lumen, or to an increase in pressure in the body cavity/lumen, when catheter system 400 is configured to expel fluids or excess fluids therefrom. Additionally/alternatively, the occlusion may lead to a decrease in the flow rate of fluid through catheter 402. Thus, according to some embodiments, sensor 406 is, or includes, a pressure sensor configured to measure/monitor pressure in catheter 402 and/or in a body cavity/lumen whereat catheter 402 is configured to be implanted (e.g. in a brain ventricle in embodiments wherein catheter system 400 is configured for draining CSF fluids from a brain ventricle). According to some embodiments, sensor 406 is, or includes, a flowmeter configured to measure/monitor the rate of flow of fluid through catheter 402.

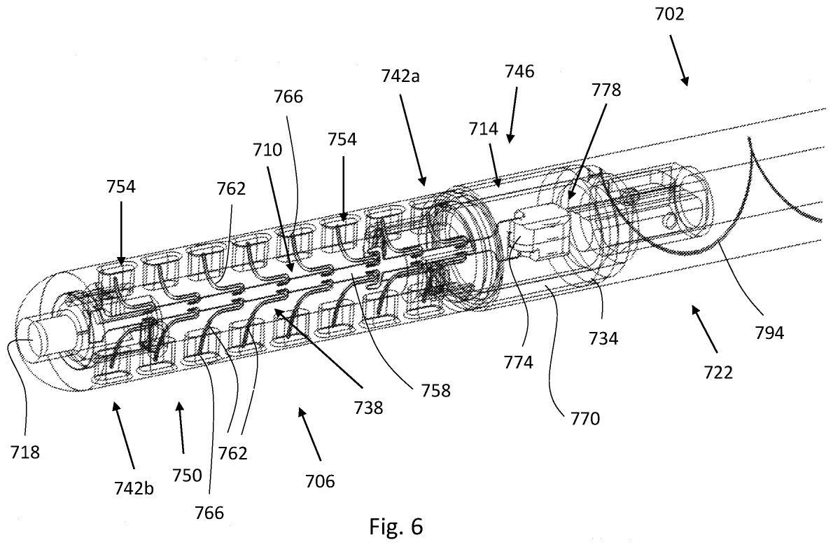

According to some embodiments, sensor 406 is positioned in/on catheter 402 (e.g. embedded in/on the walls thereof), for example, near a distal end of catheter 402 or at the distal tip thereof (e.g. essentially similarly to the sensor depicted in FIG. 6). According to some embodiments, not depicted in FIG. 4, catheter system 400 further includes a valve and/or a pump connected to catheter 402 (at a proximal end thereof) and configured to evacuate (remove) fluid therefrom (passively in the case of the valve and actively in the case of the pump) to a biological drain (e.g. the abdominal cavity). According to some such embodiments, sensor 406 is incorporated in the valve/pump.

According to some embodiments, sensor 406 is not included in catheter 402 and is configured to be implanted independently therefrom. In such embodiments, sensor 406 and control circuitry 418 may be configured for wireless communication there between (e.g. via Bluetooth). According to some such embodiments, sensor 406 may be provided independently of the rest of the components of catheter system 400.

Control circuitry 418 may be configured to analyze sensor 406 data (readings) to determine whether the data is indicative of catheter 402 being occluded, and, more generally, according to some embodiments, whether the data is indicative of a medical condition requiring attention, e.g. accumulation of excess fluid in the body cavity. More specifically, control circuitry 418 may be configured to command cleaning unit 410 to initiate a cleaning session, when sensor 406 readings indicate crossing of a predetermined threshold(s) and/or (sudden) change in a measured value. For example, when the measured pressure exceeds a pressure threshold and/or increases rapidly or when the measured flow rate through catheter 402 drops below a flow rate threshold and/or drops rapidly.

Alternatively, according to some embodiments, control circuitry 418 may be configured to communicate to external controller 500 that a cleaning session is required (instead of automatically instructing cleaning unit 410 to initiate a cleaning session), when sensor 406 readings indicate crossing of a predetermined threshold(s) and/or (sudden) change in a measured value. External controller 500 (via user interface 512) is configured to then notify the subject (or caretaker thereof) that a cleaning session is required. Control circuitry 418 may further be configured to analyze the data to estimate when a cleaning session will be required and communicate the time-estimate to external controller 500.

According to some embodiments, e.g. when external controller 500 is a mobile communication device, external controller 500 may be configured to communicate to other computational devices, mobile or otherwise, that a cleaning session is required. Thus, for example, external controller 500 may be a mobile communication device of the subject, and the other computational devices may belong e.g. to a caretaker of the subject and/or family members thereof. More specifically, according to some embodiments, in particular, embodiments wherein external controller 500 is a mobile communication device, external controller 500 may include software with similar features to the app described above in the description of mobile communication device 300. In particular, the app may be configured to independently construct a cleaning session schedule, as described above. According to some embodiments, control circuitry 118 may further be configured to communicate to external controller 500 when a cleaning session has been completed, and external controller 500 may be configured to communicate this information to the other mobile communication devices.

According to some embodiments, catheter system 400 includes a plurality of implantable sensors, e.g. a pressure sensor and a flowmeter, and the determination of whether a cleaning session is required may be performed taking into account the readings of all of the sensors. For example, according to some embodiments, the valve/pump may include a flow sensor, and the distal end of catheter 402 may include a pressure sensor. According to some embodiments, the valve/pump may include a pressure sensor, a temperature sensor, and/or a pH sensor (optionally, additionally to a flow sensor).

According to some embodiments, micro-controller 404 may be further associated with an additional sensor, which is implantable but which does not form part of catheter system 400. In such embodiments, micro-controller 404 may be wirelessly associated with the additional sensor.

According to some embodiments, sensor 406 may be configured to measure at predeterminded time intervals, e.g. every 6 hours, 8 hours, 12 hours, or once a day (with control circuitry 418 being configured to process the sensor 406 readings on receipt thereof). According to some other embodiments, sensor 406 may be configured to measure automatically upon initiation of a cleaning session.

According to some embodiments, sensor 406 may be, or may include, a temperature sensor configured to measure/monitor temperature in the body cavity. According to some embodiments, sensor 406 may be, or may include, a pH sensor configured to measure/monitor acidity levels in the body cavity.

According to some embodiments, the readings of sensor 406 may also provide indication of malfunctioning of cleaning unit 410. For example, blockage/partial blockage resultant from malfunctioning of cleaning unit 410 may lead to an increase in pressure or a decrease in flow rate. Thus, according to some embodiments, control circuitry 418 may be configured to process sensor 406 readings to determine whether cleaning unit 410 is malfunctioning. For example, if following a cleaning session, new readings of sensor 406 indicate that the occlusion has not been removed or at least mitigated, then control circuitry 418 may trigger an alert (i.e. instruct external controller 500 to generate an alert) signaling that cleaning unit 410 may be malfunctioning and that medical intervention may be required.

According to some embodiments, control circuitry 418 and cleaning unit 410 may be configured to allow controllably modifying parameters characterizing the operation of cleaning unit 410, such as the power supplied to cleaning unit 410, the duty cycle of cleaning unit 410, the activation waveform of cleaning unit 410 (e.g. the amplitude of oscillations of cleaning unit 410), etc. In such embodiments, if following a cleaning session, new readings of sensor 406 indicate that the occlusion has not been removed or at least mitigated, then control circuitry 406 may (i) initiate a corrective action including modifying one or more of the paramaters listed above, and (ii) launch a second cleaning session. The alert may be triggered if following the second cleaning session the occlusion still persists.

Similarly, according to some embodiments, control ciruitry 418 and sensor 406 are configured to allow controllably modifying parameters characterizing the operation of sensor 406, such as the sampling rate and/or the sensitivity of sensor 406.

According to some embodiments, the processing of sensor 406 readings may be performed by external controller 500. According to some embodiments, the processing may be divided between catheter system 400 and external controller 500, i.e. some of the processing may be performed by control circuitry 418 and some may be performed by processing circuitry 504.

According to some embodiments, catheter systems 100 and 400 are ventricular catheter systems for draining fluids from ventricles, in particular, cerebrospinal fluid (CSF) from brain ventricles.

FIG. 5 is a schematic, perspective view of a catheter system 600, according to some embodiments. Catheter system 600 is a specific embodiment of catheter system 100. Catheter system 600 includes a catheter 610, a casing 620 (housing electronic circuitry and power supply components as elaborated on below), and, optionally, a flexible extension 630 (e.g. a tube/cable) associating catheter 610 and casing 620, as elaborated on below. Catheter 610 is a specific embodiment of catheter 102 and includes an elongated catheter tube 702, a catheter tip member 706, a cleaning unit 710 (shown in FIGS. 6-8C), and a vibration generator 714 (shown in FIGS. 6 and 7). According to some embodiments, and as depicted in FIG. 6, catheter system 610 further includes a sensor 718 configured for occlusion detection. Cleaning unit 710, vibration generator 714, and sensor 718 are specific embodiments of cleaning unit 110, motion generator 114, and sensor 106, respectively. According to some embodiments, both casing 620 and flexible extension 630 are also implantable. It is noted that, in accordance with some embodiments, flexible extension 630 and/or casing 620 may be detachable and may be connected to catheter 610 (e.g. via a port having an electrical connector; not shown) before or after the implantation of catheter 610. According to some such embodiments, catheter system 600 may be provided with flexible extensions 630 of various lengths, to accommodate different head dimensions. For example, shorter flexible extensions may be used when catheter system 600 is implanted in children and longer flexible extensions may be used when catheter system 600 is implanted in adults.

According to some embodiments, catheter system 600 is a ventricular catheter system for draining cerebrospinal fluid from a brain ventricle, and catheter 610 is configured to be implanted in a brain ventricle. According to some such embodiments, both casing 620 and flexible extension 630 are implantable beneath the skin but outside the skull. According to some other such embodiments, casing 620 is implantable beneath the skin but outside the skull, while flexible extension 630 is implantable (under the skull but) outside the ventricle.

FIG. 6 is a schematic, perspective view of a tube distal section 722 (i.e. the distal section of catheter tube 702) and catheter tip member 706, according to some embodiments. To facilitate the description, the shells of tube distal section 722 and catheter tip member 706 are outlined but otherwise depicted as transparent, so that internal components (e.g. cleaning unit 710) are visible therein. FIG. 7 is a schematic perspective view of cleaning unit 710 and vibration generator 714.

Catheter tube 702 extends from a tube proximal end 726 (shown in FIG. 5) to a tube distal end 730. Tube proximal end 726 may be configured to be connected to a valve 732 (shown in FIG. 10), which may be similar to valve 39, as elaborated on below. Tube distal end 730 is joined to catheter tip member 706, as elaborated on below.

Catheter tip member 706 is hollow (as seen in FIG. 6) and is open at least on a tip member proximal end 734 (i.e. the proximal end of catheter tip member 706), thereby being fluidly connected to catheter tube 702. According to some embodiments, catheter tip member 706 may be tubular or in the form of a short tube. Catheter tip member 706 includes a top surface 738, a bottom surface (not shown), a first side surface 742a adjacent to both top surface 738 and to the bottom surface, and a second side surface 742b opposite to first side surface 742a.

Catheter tip member 706 further includes a tip member proximal section 746 (i.e. a proximal section of catheter tip member 706; the proximal section including tip member proximal end 734) and a tip member distal section 750 (i.e. a distal section of catheter tip member 706). Tip member proximal section 746 and tip member distal section 750 are joined.

Tip member distal section 750 includes apertures 754 (not all of which are numbered) wherethrough fluids can (i) enter catheter tip member 706 from outside thereof, when the catheter is utilized for fluid drainage/passage, and (ii) exit catheter tip member 706 to the outside thereof, when the catheter is utilized for fluid delivery/passage. Tip member proximal end 734 is connected to tube distal end 730, thereby fluidly connecting apertures 754 to catheter tube 702 and allowing to (i) expel, via catheter tube 702, fluids (e.g. CSF from a brain ventricle) drained through apertures 754, or (ii) deliver, via catheter tube 702 and apertures 754, fluids (e.g. medication) to a target site/location within a subject's body. According to some embodiments, and as depicted in the figures, apertures 754 are arranged in two rows of apertures: a first row and a second row (not numbered). The two rows may extend along the length of tip member distal section 750 on opposite sides thereof, as depicted, for example, in FIG. 6, i.e. on first side surface 742a and second side surface 742b, respectively. In other embodiments, not depicted in the figures, the apertures may be arranged in a single row, in more than two rows, or in any other applicable distribution along the length of tip member distal section 750. According to some embodiments, apertures 754 may be round. According to some embodiments, apertures 754 may be elongated, e.g. in the form of slots.

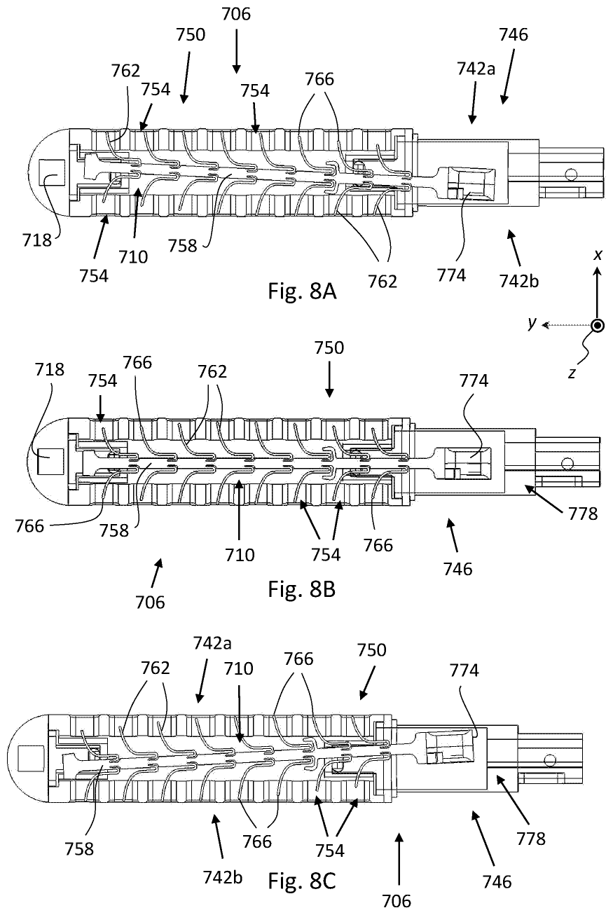

FIG. 7 is a schematic, perspective view of cleaning unit 710 and vibration generator 714, according to some embodiments. Cleaning unit 710 (depicted also in FIGS. 6 and 8A-8C) may be at least partially housed within tip member distal section 750. According to some embodiments, cleaning unit 714 includes a central shaft 758 and arms 762 (not all of which are numbered) extending from shaft 758, as disclosed, for example, in U.S. Pat. No. 9,393,389, titled “Self Cleaning Shunt”, to Samoocha et al., which is incorporated herein by reference in its entirety. According to some embodiments, arms 762 include two sets of arms: a first set and a second set (not numbered). According to some embodiments, shaft 758 and arms 762 span or substantially span a plane (e.g. shaft 758 and arms 762 lie or substantially lie in parallel to the xy-plane in FIG. 8A).

According to some embodiments, shaft 758 is longitudinally or substantially longitudinally disposed within catheter tip member 702. That is, shaft 758 may be disposed or substantially disposed in parallel to the y-axis (at least when cleaning unit 710 is not vibrating). (The coordinate system depicted in FIGS. 8A-8C should be understood to be “affixed” to catheter tip member 706, in the sense that if catheter tip member 706 is moved and/or rotated, the coordinate system is moved and/or rotated with catheter tip member 706. In particular, the axes of the coordinate system should not be construed as indicating a direction with respect to the ground (e.g. the y-axis should not be construed as being parallel to the ground).) According to some embodiments, arms 762 may be capable of projecting from shaft 758 such that tips 766 (indicated in FIGS. 7-9) of arms 762 reach into apertures 754. According to some embodiments, arms in the first set are positioned such as to allow each of the arms to extend into a respective aperture from the first row of apertures (e.g. the distances between adjacent arms in the first set equal or substantially equal the distances between adjacent apertures in the first row), and arms in the second set are positioned such as to allow each of the arms to extend into a respective aperture from the second row of apertures.

Making reference also to FIGS. 8A-8C, according to some embodiments, shaft 758 may be configured for motion/oscillation along and/or about a longitudinal axis of catheter tip member 706. (The longitudinal axis runs parallel to the y-axis.) Arms 762 may be configured for movement (e.g. of tips 766) within apertures 754 such as to prevent tissue from entering/blocking apertures 754 and/or to remove/clear/push-out tissue which has entered/blocked one or more of apertures 754 (when catheter 610 is implanted in a ventricle, for example). According to some embodiments, shaft 758 is configured for movement (e.g. vibration) such as to induce movement of arms 762/tips 766 within apertures 754. The movement of each of arms 762/tips 766 may be such as to range over all the area of the respective aperture, so as to ensure that tissue does not penetrate into the aperture. In particular, shaft 758 may be configured for oscillatory tilting motion (illustrated in FIGS. 8A-8C), so as to effect radial movement of arms 762 within apertures 754, wherein the depth of penetration of an arm into a respective aperture alternately increases and decreases. The tilting motion may be configured to occur, or substantially occur, on a plane parallel to the xy-plane. FIG. 8A depicts a stage in cleaning unit 710 oscillatory tilting motion wherein the (distal) tip of shaft 758 is displaced towards first side surface 742a. FIG. 8B depicts a stage in cleaning unit 710 oscillatory tilting motion wherein shaft 758 is disposed along the longitudinal axis of catheter tip member 706 (and is therefore not displaced). FIG. 8C depicts a stage in cleaning unit 710 oscillatory tilting motion wherein the tip of shaft 758 is displaced towards second side surface 742a. According to some embodiments, the length(s) of arms 762 is determined according to the thickness of the walls (not numbered) of tip member distal section 750 such that tips 766 do not (e.g. cannot) protrude out of tip member distal section 750, particularly, when cleaning unit 710 vibrates. According to some other embodiments, the length(s) of arms 762 is determined such that at least some of tips 766 protrude out of tip member distal section 750 when cleaning unit 710 vibrates.

According to some embodiments, arms from the first set and the second set extend into apertures from the first row and the second row, respectively, thereby suspending cleaning unit 710 within catheter tip member 706 (e.g. tips 766 remain within apertures 754, in particular, when cleaning unit 710 is activated). That is, apertures 754 support cleaning unit 710 within catheter tip member 706. Further, movement of cleaning unit 710 within catheter tip member 706 is restricted, since the movement of tips 766 is restricted by the dimensions of apertures 754.

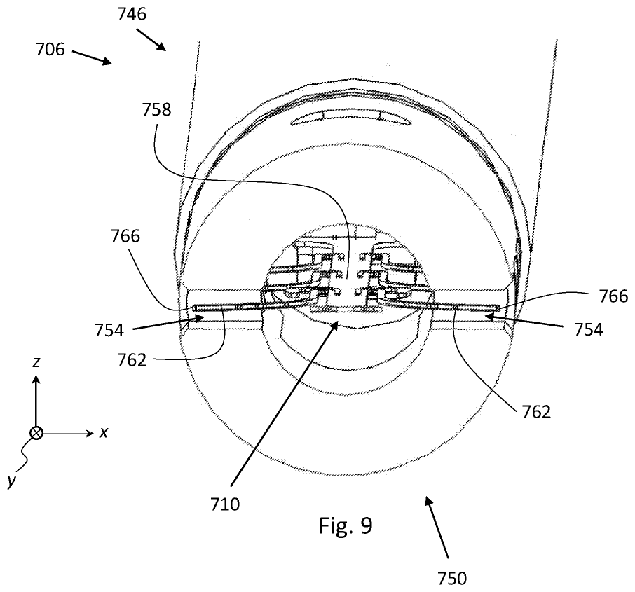

FIG. 9 provides a cross-sectional view of catheter tip member 706. The cross-section is taken along a plane perpendicular to the longitudinal axis of catheter tip member 706 (i.e. parallel to the zx-plane) such as to intersect tip member distal section 750. As shown in FIG. 9, a cross-section of each of tips 766 is smaller than respective aperture (from apertures 754) into which the tip projects. According to some embodiments, each of arms 762/tips 766 is characterized by a transverse cross-sectional area (perpendicular to the length of the respective arm) measuring at most three quarters of the corresponding area of the corresponding aperture, one half of the area of the corresponding aperture, one fourth of the area of the corresponding aperture, or even one tenth of the area of the corresponding aperture. Each possibility corresponds to a separate embodiment. According to some embodiments, each of arms 762 is characterized by a longitudinal cross-sectional area (perpendicular to the width of the respective arm (the cross-sections being parallel to the zx-plane)) measuring at most three quarters of the corresponding area of the corresponding aperture, one half of the area of the corresponding aperture, one fourth of the area of the corresponding aperture, or even one tenth of the area of the corresponding aperture. Each possibility corresponds to a separate embodiment. In particular, the shape and dimensions of arms 762 are such as to allow for unimpeded, or substantially/effectively unimpeded, fluid flow through apertures 754, at least when cleaning unit 710 is at rest.

Vibration generator 714 (e.g. an electromagnet or an electric or electromechanical motor) is configured to induce movement/vibration of shaft 758 (and arms 762). According to some embodiments, vibration generator 714 is mechanically coupled to cleaning unit 710. According to some embodiments, vibration generator 714 forms part of cleaning unit 710. According to some embodiments, and as depicted, in FIGS. 6-8C, some components of vibration generator 714 form part of catheter tip member 706 and other components of vibration generator 714 form part of cleaning unit 710. According to some embodiments, vibration generator 714 is an electromagnet including a coil 770 (of an electrically conducting wire) and a metallic casing 774. Metallic casing 774 may be or include a magnet (e.g. a neodymium magnet) and/or a magnetizable material, and may be housed in a chamber 778 inside tip member proximal portion 706. According to some embodiments, the magnet is enclosed in a corrosion-resistant metallic (e.g. titanium) casing and/or is coated with a biocompatible material. Coil 770 may be winded (wound) about a wall (not numbered, e.g. externally on the wall) of chamber 778. According to some embodiments, coil 770 is coated by an electrically-insulating material, e.g. a silicone coating or a parylene coating, or may be covered by a distal portion of catheter tube 702. Metallic casing 774 may be attached to a proximal end (not numbered) of shaft 758 such as to be at least partially disposed within coil 770.

Making reference again to FIG. 6, according to some embodiments, and as depicted in FIG. 6, sensor 718 is housed in catheter tip member 706. More specifically, according to some embodiments, sensor 718 is positioned in tip member distal section 750 at or near the distal end of catheter tip member 706, which may be open. According to some such embodiments, sensor 718 is a pressure sensor and/or a temperature sensor configured to measure/monitor intracranial pressure and/or temperature. According to some embodiments, not depicted in FIG. 6, sensor 718 may be a flowmeter positioned in tip member proximal section 746 or in catheter tube 702 and configured to measure/monitor fluid flow rate therethrough. According to some embodiments, catheter system 600 may include a plurality of sensors, e.g. a pressure sensor and a flowmeter, as described above. Casing 620 includes a printed circuit board (PCB) 780, which is a specific embodiment of control circuitry 118, and a power receiver 782 (which is a specific embodiment of power receiver 108) which includes a second coil 784 of conducting wire, which, as depicted in FIG. 5, may be flat. According to some embodiments, casing 620 further includes a wireless communication unit (e.g. a Bluetooth or an RF antenna communicatively associated by wire with PCB 780; not shown) configured to communicatively associate PCB 780 with an external activation unit, such as external activation unit 200 and specific embodiments thereof described in the description of FIGS. 11-14. According to some embodiments, the communication unit is configured to send sensor 718 readings to the external activation unit which is configured to process sensor 718 readings to determine e.g. whether catheter 610 is (potentially) at least partially blocked and/or whether a cleaning session is required. According to some embodiments, in addition to providing power, second coil 784 also serves as a transmitter and/or a receiver for communicatively associating PCB 780 with the external activation unit.

Flexible extension 630 extends from an extension proximal end 786 (the proximal end of flexible extension 630) thereof to an extension distal end 788 (the distal end of flexible extension 630). Extension proximal end 786 is connected to casing 620, either fixedly or detachably. Extension distal end 788 may be connected to catheter tube 702, such as to form a Y-junction 790 therewith. According to some embodiments, flexible extension 630 is detachably connected to catheter tube 702.

According to some embodiments, and as depicted in FIG. 5, an electrical wire(s) 794 (e.g. a co-axial cable) extends from casing 620 along flexible extension 630 and along tube distal section 722 to catheter tip member 706. According to some embodiments, at least along tube distal section 722, electrical wire(s) 794 is embedded within the walls of catheter tube 702. According to some such embodiments, at least along tube distal section 722, electrical wire(s) 794 is winded within the walls thereof. Electrical wire(s) 794 is electrically coupled to coil 770 and to sensor 718, on the distal end thereof (not numbered) and to second coil 784 and PCB 780 on the proximal end thereof (not numbered). Electrical wire(s) 794 is configured to supply electrical current to power vibration generator 714 and sensor 718, and to relay signals from sensor 718 to PCB 780, and, optionally, to relay commands from PCB 780 to sensor 718, as elaborated on below.

According to some embodiments, not depicted in the figures, instead of electrical wire(s) 794, or in addition thereto, a flexible PCB strip extends from casing 620 along flexible extension 630 and along tube distal section 722 to catheter tip member 706. The PCB strip includes conductive tracks (e.g. copper or gold tracks) electrically coupled to coil 770 and to sensor 718, on the distal end thereof and to second coil 784 and PCB 780 on the proximal end thereof. According to some embodiments, wherein instead of electrical wire(s) 794 catheter system 600 includes the PCB strip, the PCB strip is used similarly and to the same end as electrical wire(s) 794 (e.g. to power vibration generator 714 and sensor 718).

According to some embodiments, not depicted in the figures, catheter system 600 does not include flexible extension 630. Instead, casing 620 may be housed within valve 732, or positioned in proximity thereto (e.g. on catheter tube 702 proximately to tube proximal end 726), thereby obviating the need for flexible extension 630.

Vibration generator 714 may be activated by inducing an oscillating magnetic field through second coil 784, such as to induce an alternating current via second coil 784 and electrical wire(s) 794. The alternating current induces an oscillating magnetic field through coil 770 (in catheter tip member 706), which in turn induces mechanical oscillations of metallic casing 774 and cleaning element 710.

According to some embodiments, not depicted in the figures, vibration generator 714 is or includes a piezoelectric motor, which is mechanically coupled to cleaning unit 710. According to some such embodiments, the piezoelectric motor is not housed in catheter tip member 706, instead being positioned more proximally. According to some such embodiments, the piezoelectric motor is housed in a compartment located at or near Y-junction 790, and is mechanically associated with cleaning unit 710 via a mechanical infrastructure extending through tube distal section 722 and configured to impart the motion of piezoelectric motor to cleaning unit 710. The mechanical infrastructure may include, for example, a resilient rod/wire (the wire may be similar, or mechanically similar, to a guidewire). According to other such embodiments, the piezoelectric motor is housed in or near casing 620 and is mechanically coupled to cleaning unit 710 via a mechanical infrastructure as described above (the infrastructure extending also through flexible extension 630). According to some alternative embodiments, the piezoelectric motor is housed in tube distal section 722 near tube distal end 730, or in tip member proximal section 746.

According to some embodiments, wherein catheter 610 is configured to be implanted in a brain ventricle, catheter tip member 706 is characterized by a diameter between about 2 mm and about 4 mm.

According to some embodiments, catheter tip member 706 is integrally formed. According to some embodiments, catheter tip member 706 includes, or is made of, a corrosion resistant, non-toxic, and/or non-magnetic material such as titanium.

According to some embodiments, tip member distal section 750 and tip member proximal section 746 are manufactured separately as two connectable parts (which, once assembled, are not detachable). According to some embodiments, tip member proximal section 750 and tip member distal section 746 may be connected via a snap-fit mechanism (not shown). Alternatively, tip member proximal section 750 and tip member distal section 746 may be joined by welding. According to some embodiments, both tip member distal section 750 and tip member proximal section 746 include, or are made of, a corrosion resistant, non-toxic, and/or non-magnetic material, such as titanium. According to some embodiments, at least one of tip member distal section 750 and tip member proximal section 746 includes, or is made of, a polymeric material such as silicone. According to some embodiments, tip member proximal section 746 is made of titanium and covered with a silicone cover: over coil 770 and proximally therefrom. The silicone cover may constitute a distal portion of catheter tube 702 or constitute a dedicated silicone coating. The silicone cover may be impregnated with antibiotics, hydrophilic or hydrophobic, barium, and/or other materials as commonly used in implanted catheters.

According to some embodiments, additionally or alternatively to sensor 718, catheter system 600 may include a motion sensor configured to detect motion of cleaning unit 710 and to output to a signal(s) indicative of the motion to PCB 780. PCB 780 may be configured to analyze (process) the signal(s) to determine whether cleaning unit 710 is working properly, or PCB 780 may be configured to forward the signal(s) to external processing circuitry (e.g. in an external activation unit such as external activation unit 200 or in an external controller such as external controller 500), the external processing being configured to analyze the signal(s) to determine whether cleaning unit 710 is working properly.

In particular, in embodiments wherein the motion of cleaning unit 710 is reciprocal/oscillatory, the signal(s) may be processed to compute the amplitude of the motion of cleaning unit 710 and/or the mean (average) position of the cleaning unit 710: A small amplitude may be indicative of limited motion due to blockage, e.g. of one or more of apertures 754, and/or a malfunction in cleaning unit 710 (or in other components associated thereto). A mean position which is displaced relative to the “normal” mean position (that is the mean position when cleaning unit 710 is working properly) may be indicative of unilateral blockage or partial blockage. According to some embodiments, if the analysis of the signal(s) indicates a malfunction, a corrective action may be initiated to rectify the malfunction. The corrective action may include increasing a power supplied to the cleaning unit, changing a duty cycle of the cleaning unit, and/or changing an activation waveform of the cleaning unit.

According to some such embodiments, the motion sensor may be an optical sensor and/or a proximity sensor. According to some embodiments, wherein vibration generator 714 is an electromagnet, the motion sensor may be a magnetic sensor (e.g. a Hall effect sensor) configured to detect motion of metallic casing 784. In such embodiments, the motion sensor may be positioned in proximity to metallic casing 784, e.g. at, or near, tube distal end 730.

According to some embodiments, a mandrel may be used to implant catheter 610, and, in particular, to guide catheter tip member 706 to an intended implantation site (e.g. within a ventricle.) According to such some embodiments, not depicted in the figures, catheter tip member 706 further includes a stopper configured to be engaged by a tip portion of the mandrel, such as to prevent the mandrel from at least one of reaching and damaging cleaning unit 710 during the implantation of catheter 610. According to some embodiments, the stopper may include a first geometrical feature (e.g. an inwardly extending flange) projecting from an inner surface of tip member proximal section 746 and the tip portion of the mandrel may include a second geometrical feature (e.g. a flange or band) radially projecting relative to a main body of the mandrel. The second geometrical feature is configured to engage the first geometrical feature, such as to allow guiding catheter tip member 706 using the mandrel.

According to such some embodiments, the stopper includes a first key pattern and the tip portion of the mandrel includes a second key pattern complementary to the first key pattern. The first and second key patterns may be configured to interlock, upon engaging of the stopper by the tip portion of the mandrel, such that a rotation of the mandrel induces an equal rotation of the catheter tip member. According to some such embodiments, the first key pattern may be configured as male and the second key pattern may be configured as female, or the first key pattern may be configured as female and the second key pattern may be configured as male.

According to some embodiments, not depicted in the figures, instead of central shaft 758, cleaning unit 710 includes an elongated rod and a rigid sleeve slidably mounted on the rod. The rod may extend along the axial axis of catheter tip member 706 and may be fixed both on a proximal end thereof and on a distal end thereof (e.g. to the distal end of catheter tip member 706). In such embodiments, the arms of the cleaning unit are connected to the sleeve such that when the sleeve is distally slid on the rod, the arms project into the respective apertures (from apertures 754). According to some embodiments, the sleeve is switchable between at least two configurations. In a first configuration the arms are folded on the sleeve, such that the arms do not, or substantially do not, radially project from the sleeve. In the second configuration the sleeve is distally positioned relative to the first configuration, and the arms radially project from the sleeve into the apertures. According to some embodiments, in switching from the first configuration to the second configuration, the arms project in a manner similar to the arms of an umbrella when the umbrella is opened. According to some embodiments, the sleeve may be switched to the second configuration after cleaning unit 710 has been positioned within catheter tip member 706, but before catheter 702 has been implanted within the subject's body, e.g. during assembly or by the medical personnel. According to some embodiments, the sleeve may be switched to the second configuration after catheter 702 has been implanted. According to some embodiments, at least some of the movement of the arms in the apertures may be effected by slightly pushing and pulling the sleeve when the sleeve is in the second configuration, such as to induce a small amplitude oscillatory motion of the sleeve along the rod. According to some embodiments, instead of a rigid sleeve, the rod may have slidably mounted thereon one or more rings, each of the rings having connected thereto one or more (e.g. a pair) of the arms.

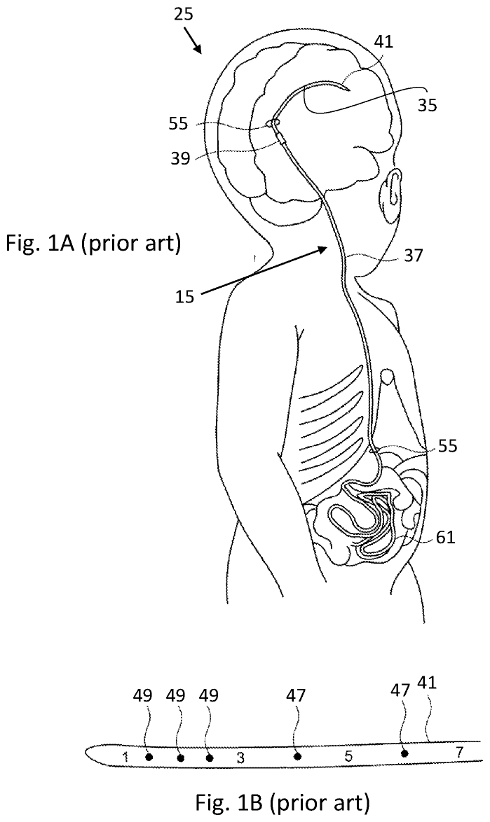

Making reference to FIG. 10, FIG. 10 is a perspective view of a catheter assembly 800 for draining body fluids, including catheter system 600 and a flexible drain tube 810, similar to drain tube 37. Drain tube 810 is fluidly coupled on an end thereof, via valve 732, to tube proximal end 726. In operation, once catheter assembly 800 is implanted in a patient (essentially as depicted in FIG. 1A), body fluids are drained via apertures 754. According to some embodiments, e.g. wherein catheter 610 is implanted in a brain ventricle and the body fluids are CSF, drained fluids may travel in the proximal direction from catheter tip member 706 into catheter tube 702, and therefrom via drain tube 810 into e.g. an abdominal cavity of the patient. More specifically, valve 732 regulates the flow of fluid from catheter tube 602 into drain tube 810. Valve 732 may be a one-way valve thereby ensuring that fluid can only flow from catheter tube 702 to drain tube 810 and not in the opposite sense (or, only in the opposite sense (direction), in fluid delivery applications). According to some embodiments, cleaning unit 710 may be activated on a regular basis (e.g. for five minutes once a day), either manually or automatically, to ensure that apertures 754 do not become blocked by cell growth.

According to some embodiments, wherein casing 620 and flexible extension 630 are implantable, an external activation unit may be provided. The external activation unit may be configured to generate an oscillating magnetic field, so that, when operated e.g. by a patient (i.e. a subject) or a caregiver, the generated magnetic field induces an alternating current via second coil 784. FIG. 11 schematically depicts such an exemplary external activation unit 900 in the form of a headset 902 configured to be worn on the head of a subject, according to some embodiments. External activation unit 900 is a specific embodiment of external activation unit 200 of FIG. 2. More specifically, FIG. 11 schematically depicts a subject implanted with catheter assembly 800 (such that catheter tip member 706 is disposed within a brain ventricle) and wearing headset 902 on a head 950 of the subject. Power receiver 782 is shown implanted beneath a skin 956 on head 950 and outside a skull 958 of the subject.

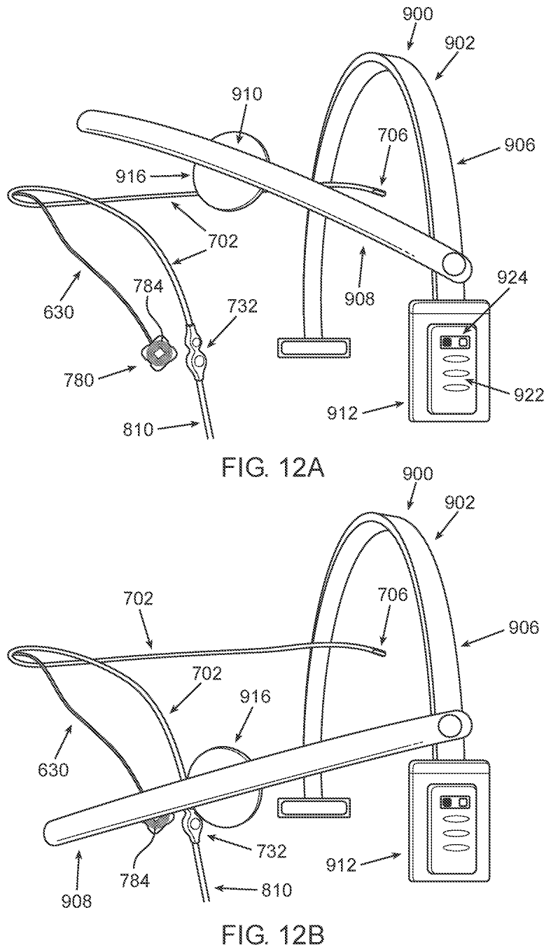

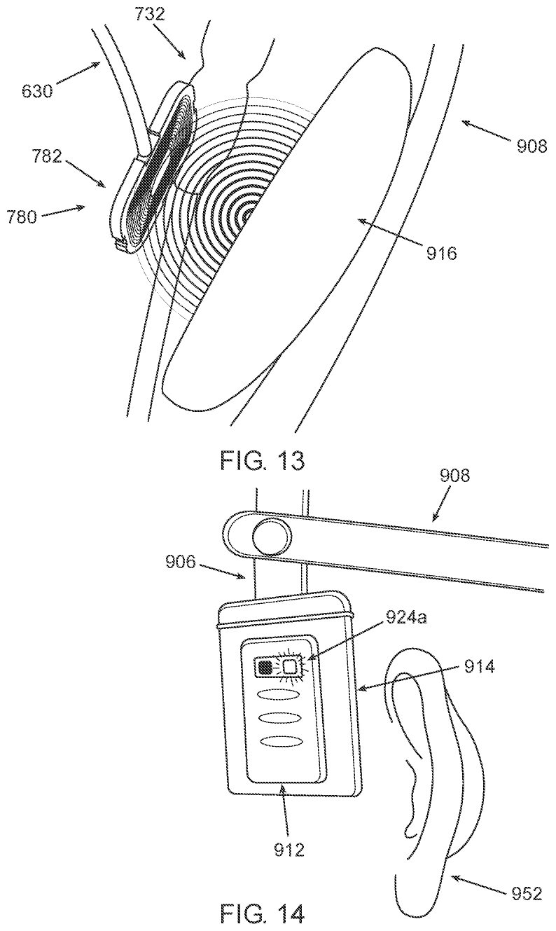

According to some exemplary embodiments, headset 902 includes an adjustable band 906, configured to secure headset 902 to head 950, and an arm 908. Headset 902 includes processing circuitry, a communication unit (both not shown), and a user interface 912, which are specific embodiments of processing circuitry 204, receiver 208, and user interface 212. According to some embodiments, the processing circuitry, communication unit, and user interface 912 may be housed together or separately in band 906 and/or on arm 908 at any location along band 906 and arm 908. According to some embodiments, the processing circuitry and the communication unit may be housed within a casing 914. According to some embodiments, and as depicted in FIG. 11, user interface 912 may be installed on casing 914. Arm 908 includes a power transmitter 916, which is a specific embodiment of power transmitter 216. According to some embodiments, power transmitter 916 may be positioned on an arm portion 910 (which may be, for example, centrally located on arm 908). According to some embodiments, the position of power transmitter 916 may be adjustable, i.e. power transmitter 916 can be moved back and forth along the length of arm 908, so as to ensure alignment between power transmitter 916 and the implanted casing 620 (specifically, with power receiver 782) when headset 902 is worn on the head of a subject (thereby allowing to account for different head sizes (e.g. due to age) and different implant locations of casing 620). Band 906 may further include a replaceable and/or rechargeable battery (not shown). According to some embodiments, headset 902 may be connected to an external power source for charging the battery. According to some embodiments, headset 902 is configured to not allow WPT when being charged. According to some embodiments, headset 902 is configured to not allow the wearing thereof when connected to the external power source (essentially as described herein above). According to some embodiments, headset 902 may be connectable to an external power source (e.g. an external battery) to power a cleaning session. According to some embodiments, and as indicated in FIG. 12A, user interface 912 includes a speaker 922 and indicator lights 924. To initiate a cleaning session, the subject puts on headset 902. According to some embodiments, wherein user interface 912 includes speaker 922, headset 902 is shaped such that when properly positioned on head 950, speaker 922 is positioned adjacently to (as depicted in FIG. 11 and in FIG. 14) or on an ear 952 of the subject, thereby facilitating the provision of audio-signals to the subject. According to some embodiments, not depicted in FIG. 11 and FIG. 14, speaker 922 is an earplug speaker. According to some embodiments, and as depicted in FIGS. 12A and 12B, arm 908 is maneuverable such as to allow positioning arm portion 910 adjacently to casing 620, so that power transmitter 916 is adjacent to power receiver 782 (as depicted in FIG. 13). For example, the position of arm 908 in FIG. 12A may be such that when headset 902 is properly put on the head of the subject, arm portion 910 is not adjacent to casing 620, whereas the position of arm 908 in FIG. 12B may be such that when headset 902 is properly put on the head of the subject, arm portion 910 is adjacent to casing 620. According to some embodiments, user interface 912 may be used to launch a cleaning session (when arm 908 is in the second configuration). Power transmitter 916 includes a coil of conducting wire (not shown), being thereby configured to transfer power to power receiver 782 via inductive coupling, so as to activate cleaning unit 710 and sensor 718, and launch a cleaning session. According to some embodiments, in the configuration of headset 902 depicted in FIG. 12A, headset 902 may be configured to prohibit transmission of power (from power transmitter 916), while in the configuration of headset 902 depicted in FIG. 12B, headset 902 may be configured to allow transmission of power.

According to some embodiments, catheter system 600 and headset 902 are configured such that the maneuvering of arm 908 to a position wherein arm portion 910 is adjacent to casing 620 (e.g. switching headset 902 from the configuration depicted in FIG. 12A to the configuration depicted in FIG. 12B) when headset 902 is properly worn, automatically activates cleaning unit 710 and launches a cleaning session.

According to some embodiments, headset 902 may be communicatively associated with a mobile communication device 1000, such as a smartphone (as depicted in FIG. 11), a tablet, or a laptop of the subject, which may be used to activate headset 902 (and thereby allow initiating a cleaning session using mobile communication device 1000). Mobile communication device 1000 is a specific embodiment of mobile communication device 300. According to some embodiments, wherein catheter system 600 includes a sensor (such as sensor 106) for detecting occlusion in catheter 610, headset 902 may be configured to transmit the sensor signals (received from catheter system 600) to mobile communication device 1000, which may include software (e.g. an app) configured to process the sensor signals to determine whether occlusion is present and/or whether a cleaning session is required. The software installed on mobile communication device 1000 may be further configured to analyze and display to the subject/caregiver (e.g. using tables, graphs, etc.) data received from a processor(s) included in PCB 780, via headset 902, such as timing of one or more recent cleaning sessions (e.g. the last 3, 5, or 10 last cleaning sessions), the durations of each of the recent cleaning sessions, correlation between the timings of the recent cleaning sessions and the changes in intracranial pressure (ICP), etc.

According to some embodiments, there is provided an external activation unit, which is a specific embodiment of external activation unit 200, configured for use with a commercial headset e.g. for listening to music. The external activation unit includes a mountable arm similar to arm 908 (and including a power transmitter similar to power transmitter 916) configured to be mounted on/removably attached to the headset. According to some embodiments, a user interface, similar to user interface 912 and associated with the power transmitter, may also be mounted on the headset. According to some embodiments, the user interface may be included in the mountable arm. According to some embodiments, the arm includes processing circuitry and a wireless communication unit and is configured to be operated using a mobile communication device, such as a smartphone.

FIG. 14 depicts headset 902 powering a cleaning session of catheter system 600, according to some embodiments. An indicator light 924a (from indicator lights 924) on user interface 912 is switched on to indicate that a cleaning session is in progress.

According to an aspect of some embodiments, there is provided a catheter system (not shown in the figures), which is similar to catheter system 600 but differs therefrom in including an implantable battery in place of power receiver 782. As such, the catheter system provides a specific embodiment of catheter system 400.

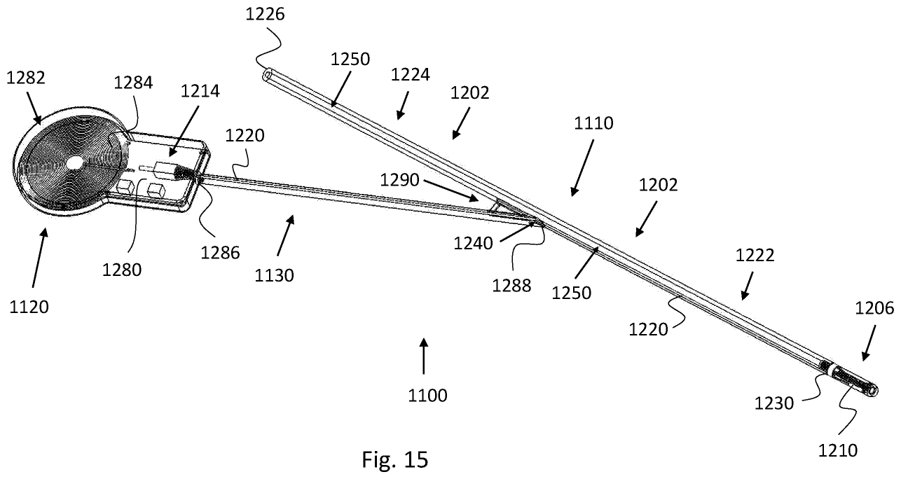

According to an aspect of some embodiments, and as depicted in FIGS. 15-17, there is provided a catheter system 1100. Catheter system 1100 is a specific embodiment of catheter system 100 and is similar to some embodiments of catheter system 600. Catheter system 1100 may be configured/designed such as to minimize the number of electronic components positioned inside the skull when catheter system 1100 is implanted in the head of a subject, particularly, the electronic components involved in supplying power and imparting motion to a cleaning unit of catheter system 1100. According to some embodiments, all the electronic components directly involved in supplying power to the cleaning unit are positioned outside the skull, or at least outside the brain, when catheter system 1100 is implanted in the head of a subject. As used herein, according to some embodiments, electronic components such as a power receiver or a battery, electrical wires or a PCB strip, a motor, and so on, are said to be “directly” involved in supplying power to the cleaning unit, whereas an occlusion sensor may be said at most to be “indirectly” involved in powering the cleaning unit (since, in principle, readings indicating occlusion may lead to the activation of the cleaning unit). According to some embodiments, all the electronic components directly or indirectly involved in supplying power and imparting motion to the cleaning unit are positioned outside the skull, or at least outside the brain, when catheter system 1100 is implanted in the head of a subject.

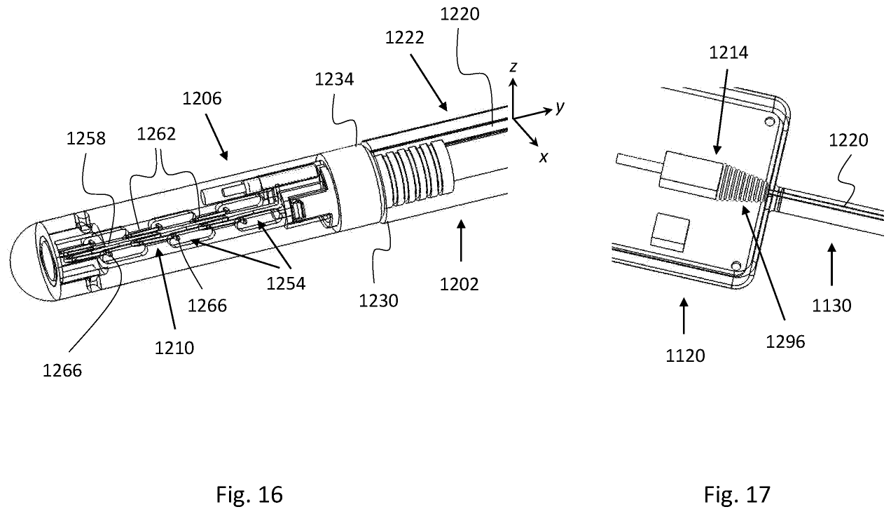

FIG. 15 is a schematic, perspective view of catheter system 1100, according to some embodiments. Catheter system 1100 includes a catheter 1110, a casing 1120, and a flexible extension 1130 associating catheter 1110 and casing 1120, similarly to the association between catheter 610 and casing 620 provided by extension 630 and described above in the description of catheter system 600. Catheter 1110 includes an elongated catheter tube 1202, a catheter tip member 1206, a cleaning unit 1210 (shown in detail in FIG. 16), and a motion actuator 1214 which, as depicted in FIGS. 15 and 17, may be housed in casing 1120. Cleaning unit 1210 and motion actuator 1214 are specific embodiments of cleaning unit 110 and motion generator 114, respectively. According to some embodiments, both casing 1120 and flexible extension 1130 are also implantable. It is noted that, in accordance with some embodiments, flexible extension 1130 and/or casing 1120 may be detachable and may be connected to catheter 1110 (e.g. via a port having an electrical connector; not shown) before or after the implantation of catheter 1110. According to some such embodiments, catheter system 1100 may be provided with a plurality of flexible extensions of various lengths (each being a specific embodiment of flexible extension 1130) to accommodate anatomical variations (i.e. different head dimensions).

According to some embodiments, catheter system 1100 is a ventricular catheter system for draining CSF from a brain of a subject. According to some embodiments, catheter 1110 is configured to be implanted in a brain ventricle. According to some embodiments, both casing 1120 and flexible extension 1130 are implantable beneath the skin but outside the skull. According to some other embodiments, casing 1120 is implantable beneath the skin but outside the skull, while flexible extension 1130 may be implantable in part inside the skull but outside the brain.

Catheter tube 1202 extends from a tube proximal end 1226 to a tube distal end 1230. Tube proximal end 1226 may be configured to be connected to a valve (not shown), such as, or similar to, valve 732 of catheter system 600. Tube distal end 1230 is joined to catheter tip member 1206, essentially similarly to the joining of tube distal end 730 and catheter tip member 706 of catheter 610.

Catheter tip member 1206 is similar to catheter tip member 706, being hollow and open at least on a tip member proximal end 1234 (indicated in FIG. 16), thereby being fluidly connected to catheter tube 1202. Catheter tip member 1206 includes apertures 1254 (shown in FIG. 16; not all of which are numbered) wherethrough CSF can enter catheter tip member 1206 from the outside thereof (e.g. from a brain ventricle). Tip member proximal end 1234 is connected to tube distal end 1230, thereby fluidly connecting apertures 1254 to catheter tube 1202 and allowing to expel, via catheter tube 1202, CSF drained through apertures 1254.

FIG. 16 is a schematic, perspective view of catheter tip member 1206, according to some embodiments. To facilitate the description, the shell of catheter tip member 1206 is outlined but otherwise depicted as transparent so that cleaning unit 1210 is visible therein. Cleaning unit 1210 may be similar to cleaning unit 710 of catheter system 600. According to some embodiments, cleaning unit 1210 includes a central shaft 1258 and arms 1262 (not all of which are numbered) extending from shaft 1258. According to some embodiments, arms 1262 include two sets of arms: a first set and a second set (not individually numbered). According to some embodiments, shaft 1258 is longitudinally or substantially longitudinally disposed within catheter tip member 1206. That is, shaft 1258 may be disposed or substantially disposed in parallel to they-axis (at least when cleaning unit 1210 is not in motion). (The coordinate system appearing in FIG. 16 should be understood to be affixed to catheter tip member 1206.) According to some embodiments, arms 1262 may be capable of projecting from shaft 1258 such that tips 1266 of arms 1262 reach into apertures 1254.

According to some embodiments, shaft 1258 may be configured for motion/oscillation along and/or about a longitudinal axis of catheter tip member 1206. Arms 1262 may be configured for movement within apertures 1254 such as to prevent tissue from entering/blocking apertures 1254 and/or to remove/clear/push-out tissue which has entered/blocked one or more of apertures 1254, essentially as described above in the description of cleaning unit 710 of catheter system 600.

Motion actuator 1214 is configured to induce movement/vibration of shaft 1258 (and arms 1262). More specifically, motion actuator 1214 is mechanically coupled to cleaning unit 1210 via an elongated extender element 1220, which is configured to transfer/impart to cleaning unit 1210 motion actuator 1214 movements/vibration. Extender element 1220 may extend from casing 1120 to/into catheter tip member 1206, via flexible extension 1130 and a tube distal section 1222 (i.e. the distal section of catheter tube 1202), and may be connected on a proximal end thereof (not numbered) to motion actuator 1214 and on a distal end thereof (not numbered) to cleaning unit 1210 (e.g. to the proximal end of shaft 1258). According to some embodiments, the distal end of extender element 1220 is soldered/welded onto the proximal end of shaft 1258. According to some embodiments, motion actuator 1214 may be configured to induce axial motion of extender element 1220 and thereby to allow inducing longitudinal, reciprocal motion of shaft 1258 within catheter tip member 1206. According to some embodiments, motion actuator 1214 may be configured to induce reciprocal motion, rotational motion, vibrational motion, oscillatory motion, axial motion, radial motion, tilting, and/or any combination thereof, and thereby to induce similar motion/movement of cleaning unit 1210. According to some embodiments, motion actuator 1214 is an electro-mechanical motor. According to some embodiments, motion actuator 1214 is a piezoelectric motor. According to some embodiments, motion actuator 1214 is or includes an electromagnet. According to some embodiments, e.g. embodiments wherein motion actuator 1214 is a piezoelectric motor, extender element 1220 is coupled to motion actuator 1214 by means of a bellows member 1296. Bellows member 1296 may be configured to repeatedly expand and constrict (shrink), when motion actuator 1214 is activated, so as to push and pull extender element 1220 back and forth, thereby inducing axial, reciprocal motion of cleaning unit 1210.

According to some embodiments, catheter tube 1202 includes a pair of lumens. A first lumen 1250 extends along the full length of catheter tube 1202 and fluidly couples catheter tip member 1206, and apertures 1254, to tube proximal end 1226. First lumen 1250 is configured for fluid passage therethrough such as to allow draining CSF from a brain ventricle, essentially as described herein above. A second lumen (not shown) extends along tube distal section 1222 in parallel to first lumen 1250 and may be fluidly decoupled from first lumen 1250. In such embodiments, extender element 1220 may extend along and through the second lumen (as well as through flexible extension 1130). Thus, flexible extension 1130 and the second lumen essentially define a duct/passage along (and inside of) which extender element 1220 is disposed; the duct/passage may be fluidly decoupled from first lumen 1250 so that CSF fluid is prevented from entering the duct. According to some embodiments, the second lumen may be embedded in the walls of tube distal section 1222.

FIG. 17 is a schematic, perspective, partial view of casing 1120 and some of the components housed therein, according to some embodiments. Making reference also to FIG. 15, in addition to motion actuator 1214, casing 1120 may further house a PCB 1280 (which is a specific embodiment of control circuitry 118), or, more generally, any other type of electronic circuitry configured to control cleaning unit 1210 operation. In particular, casing 1120 may house a micro-controller, which is a specific embodiment of micro-controller 104, and includes PCB 1280. According to some embodiments, wherein catheter system 1100 is configured to be powered by WPT, casing 1120 also includes a power receiver 1282 (which is a specific embodiment of power receiver 108). According to some embodiments, wherein power receiver 1282 is configured for WPT via inductive coupling, and as depicted in FIGS. 15 and 17, power receiver 1282 includes a coil 1284 of conducting wire. According to some embodiments, not depicted in FIGS. 15 and 17, catheter system 1100 includes an implantable battery (similar to a pacemaker battery) configured to power, or at least partially power, catheter system 1100. According to some embodiments, casing 1120 further includes a wireless communication unit (e.g. a Bluetooth or an RF antenna communicatively associated by wire with PCB 1280; not shown) configured to communicatively associate PCB 1280 with an external activation unit, such as headset 902 (depicted in FIG. 11), and/or a mobile communication device. According to some embodiments, in addition to providing power, coil 1284 also serves as a transmitter and/or a receiver for communicatively associating PCB 1280 with the external activation unit. According to some embodiments, motion actuator 1214 is positioned on PCB 1280.

Flexible extension 1130 extends from an extension proximal end 1286 (the proximal end of flexible extension 1130) to an extension distal end 1288 (the distal end of flexible extension 1130). Extension proximal end 1286 (the first end of flexible extension 1130) is connected to casing 1120, either fixedly or detachably. Extension distal end 1288 (the second end of flexible extension 1130) may be connected to catheter tube 1202, such as to form a Y-junction 1290 therewith. More specifically, Y-junction 1290 partitions catheter tube 1202 into tube distal section 1222 and a tube proximal section 1224, with tube distal section 1222 (or at least the bulk thereof) being configured to be implanted in the brain and tube proximal section 1224 (or at least the bulk thereof) being configured to be implanted outside the skull and to evacuate (remove) excess fluid from the brain to a biological drain (e.g. the abdominal cavity). According to some embodiments, flexible extension 1130 is detachably connected to catheter tube 1202.

According to some embodiments, extender element 1220 includes one or more resilient wires (e.g. similar to a guidewire) and/or rods configured to transfer/impart motion/vibration from motion actuator 1214 to cleaning unit 1210. According to some embodiments, wherein cleaning unit 1210 is configured for rotational motion/oscillations about the longitudinal axis (not indicated) of catheter tip member 1206, the torsional flexibility of extender element 1220 is such as to allow the rotational motion/oscillations. According to some embodiments, extender element 1220 may be bent. More specifically, according to some embodiments, extender element 1220 may include a bent section 1240 which is positioned at Y-junction 1290, so that the shape of extender element 1220 conforms to the shape defined by flexible extension 1130 and tube distal section 1222. According to some embodiments, extender element 1220 is assembled from two or more longitudinal elements. For example, extender element 1220 may include two co-axial elements, such as an external tube and an internal rod/wire (not shown) disposed within the external tube along the length thereof. The inner rod/wire may be configured for motion within the external tube (e.g. longitudinal, reciprocal motion) such as to transfer/impart the movements/vibrations induced by motion actuator 1214 to cleaning unit 1210. Further, the external tube may also function to minimize possible friction between the inner wire/rod and flexible extension 1130 and tube distal section 1222, particularly at Y-junction 1290, whereat, according to some embodiments, extender element 1220 may be bent (e.g. curved or partially folded). According to some embodiments, the external tube may be metallic and may include a low friction inner surface configured to reduce frictional forces exerted on the inner rod/wire.

According to some embodiments, catheter system 1100 may further include a sensor (not shown), communicatively associated with PCB 1280 and configured to monitor motion actuator 1214 operation.

According to some embodiments, not depicted in the figures, catheter system 1100 includes an occlusion sensor, which may be similar to sensor 718 of catheter system 600, and which is functionally associated with PCB 1280, respectively.

The skilled person will understand that the scope of the disclosure also covers embodiments wherein motion actuator 1214 is partially or fully positioned inside flexible extension 1130.

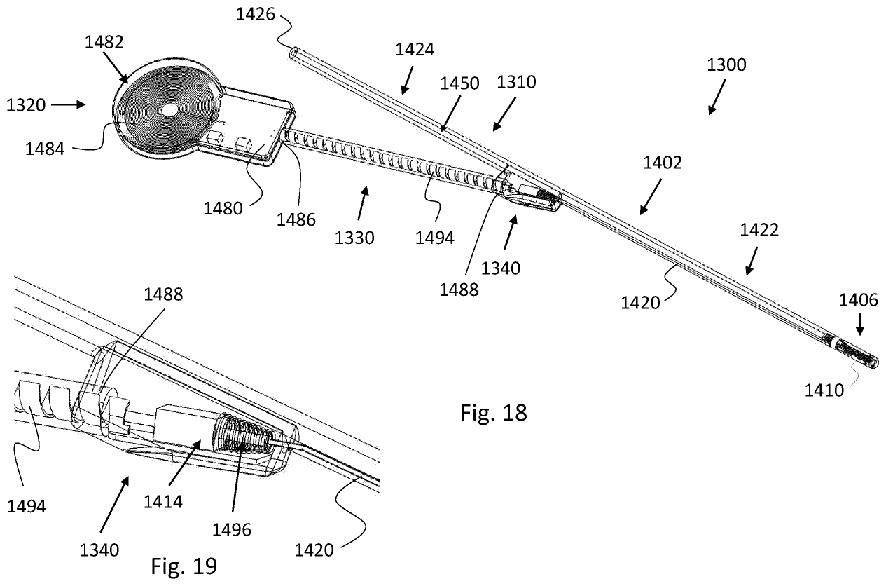

According to an aspect of some embodiments, and as depicted in FIGS. 18 and 19, there is provided a catheter system 1300. Catheter system 1300 is a specific embodiment of catheter system 100. FIG. 18 is a schematic perspective view of catheter system 1300, according to some embodiments. Catheter system 1300 includes a catheter 1310, a casing 1320, a flexible extension 1330, and a compartment 1340 housing a motion actuator 1414 (indicated in FIG. 19). Casing 1320 may house a PCB 1480 (similar to PCB 1280, and/or, more generally, other electronic circuitry) and a power supply source such as a power receiver 1482 (similar to power receiver 1282), as elaborated on below. In particular, casing 1320 may house a micro-controller, which is a specific embodiment of micro-controller 104 and includes PCB 1480.

Flexible extension 1330 extends from an extension proximal end 1486 (a first end of the extension) to an extension distal end 1488 (a second end of the extension). Extension proximal end 1486 is connected to casing 1320, either fixedly or detachably. Extension distal end 1488 is connected to compartment 1340, either fixedly or detachably.

Catheter 1310 includes a catheter tube 1402, a catheter tip member 1406, and a cleaning unit 1410 housed (at least in part) within catheter tip member 1406. Catheter tube 1402 includes a tube distal section 1422 and a tube proximal section 1424. Catheter tip member 1406 and cleaning unit 1410 may be essentially similar to catheter tip member 1206 and cleaning unit 1210 of catheter system 1100.

Tube distal section 1422 extends distally from compartment 1340, while each of flexible extension 1330 and tube proximal section 1424 extend proximally from compartment 1340, such that catheter 1310, flexible extension 1330, and compartment 1340 are arranged in a Y-shaped configuration (i.e. a Y-shaped arrangement). According to some embodiments, and as depicted in FIGS. 18 and 19, compartment 1340 is positioned adjacently to catheter tube 1402, e.g. a side wall (not numbered) of compartment 1340 may be attached to catheter tube 1402. According to some embodiments, catheter system 1300 is configured such that, when implanted in the head of a subject, compartment 1340 is positioned outside the skull. According to some other embodiments, catheter system 1300 is configured such that, when implanted in the head of a subject, compartment 1340 is positioned at least in part inside the skull (but outside the brain).

Motion actuator 1414 is a specific embodiment of motion generator 114 and is configured to induce movement/vibration of cleaning unit 1410. Motion actuator 1414 is mechanically coupled to cleaning unit 1410 via an elongated extender element 1420, which is configured to transfer/impart to cleaning unit 1410 the motion induced by motion actuator 1414. Extender element 1420 may extend from compartment 1340 to catheter tip member 1406 via tube distal section 1422. More specifically, motion actuator 1414 is configured to induce motion/vibration of a central shaft (which may be essentially similar to shaft 1258 of cleaning unit 1210; not numbered) and arms (which may be essentially similar to arms 1262 of cleaning unit 1210; not numbered) of cleaning unit 1410. According to some embodiments, motion actuator 1414 is an electro-mechanical motor. According to some embodiments, motion actuator 1414 is a piezoelectric motor. According to some embodiments, motion actuator 1414 is or includes an electromagnet. According to some embodiments, motion actuator 1414 may be coupled to extender element 1420 by a bellows member 1496 configured to repeatedly expand and contract so as to impart motion actuator 1414 motion/vibration to extender element 1420, which in turn imparts the motion to cleaning unit 1410. According to some embodiments, extender element 1420 includes one or more resilient wires and/or rods configured to transfer/impart motion/vibration from motion actuator 1414 to cleaning unit 1410.

According to some embodiments, catheter tube 1402 includes at least two lumens. A first lumen 1450 extends along the full length of catheter tube 1402 and fluidly couples catheter tip member 1406 to a tube proximal end 1426. First lumen 1450 is configured for fluid passage therethrough such as to allow draining CSF from a brain ventricle, essentially as described herein above. A second lumen (not shown) extends along tube distal section 1422 in parallel to first lumen 1450 and may be fluidly decoupled from first lumen 1450. In such embodiments, extender element 1420 may extend along (and inside) the second lumen. According to some embodiments, the second lumen may be embedded in the walls of tube distal section 1422.

According to some embodiments, and as depicted in FIG. 18, a flexible PCB strip 1494 extends along flexible extension 1330 from casing 1320 to compartment 1340. Conduction tracks (not shown), running along the length of PCB strip 1494, functionally associate motion actuator 1414 with PCB 1480 (such as to allow PCB 1480 to control operation of motion actuator 1414, e.g. switching on/off) and with power receiver 1482.

Power receiver 1482 is configured to receive power by WPT (from an external activation unit such as headset 902 depicted in FIG. 11) and to power motion actuator 1414. According to some embodiments, and as depicted in FIG. 18, power receiver 1482 is configured for WPT via inductive coupling and includes to this end a coil 1484 of conducting wire. According to some embodiments, PCB strip 1494 may be helically disposed inside flexible extension 1330. According to some embodiments, a helical arrangement renders the long PCB strip less susceptible to damage (kinks, abrasions, etc.) as compared to when arranged as a long straight strip.

The skilled person will understand that instead of PCB strip 1494, or in addition thereto, electrical wire(s) may be used (i) to implement the communication between motion actuator 1414 and PCB 1480 and/or (ii) to provide power to motion actuator 1414. The skilled person will also understand that instead of power receiver 1482, or in addition thereto, other power supply means may be used, such as an implantable battery which may be housed in casing 1320.

According to some embodiments, compartment 1340 may additionally include electronic circuitry (not shown) functionally associated with motion actuator 1414 and related to the operation thereof. The electronic circuitry may be communicatively associated with PCB 1480 via PCB strip 1494 (and/or electrical wire(s)). According to some embodiments, the electronic circuitry may include a second PCB (i.e. so that a first PCB—PCB 1480—is housed in casing 1320 and the second PCB is housed in compartment 1340). According to some other embodiments, PCB 1480 is housed in compartment 1340 and is electrically coupled to power receiver 1482 via PCB strip 1494 (and/or electrical wire(s)).

The skilled person will understand that catheter system 1300 may additionally include features/elements—which have been described with respect to catheter system 1100, but which have not been mentioned with respect to catheter system 1300—in a similar manner to their incorporation in catheter system 1100. Thus, for example, casing 1420 may include a wireless communication unit configured to communicatively associate catheter system 1300 with an external activation unit (such as external activation unit 200) and/or an external controller (such as external controller 500).

According to some embodiments, catheter system 1300 may further include a sensor (not shown), communicatively associated with PCB 1480 and configured to monitor motion actuator 1414 operation.

According to an aspect of some embodiments, not depicted in the figures, there is provided a catheter system similar to catheter systems 1100 and 1300 in including an elongated extender element but differing therefrom in that the extender element and the cleaning unit are magnetically coupled (instead of mechanically coupled). More specifically, the extender may be connected on a first (and proximal) end thereof to the motion actuator, essentially as described above with respect to catheter system 1100 or catheter system 1300. A distal portion of the extender element may be configured to be magnetizable such as to allow controllably inverting the polarity thereof, or the distal portion may include an electromagnet. The cleaning unit may also include a magnet (e.g. on a proximal portion thereof). The catheter system may be configured to switch the polarity of the distal portion of the extender element such that (i) when the extender element, or at least the distal portion thereof, is moving distally, the distal end of the extender element repels the cleaning unit, thereby imparting distal motion thereto, and (ii) when the extender element, or at least the distal portion thereof, is moving proximally, the distal end of the extender element attracts the cleaning unit, thereby imparting proximal motion thereto.

According to some embodiments, when the cleaning unit is displaced in the distal direction, the arms of the cleaning unit are pressed against the distal walls of the respective apertures, and due to the elasticity thereof, the arms act to push back the cleaning unit (in the proximal direction). In such embodiments, an alternative mechanism for the magnetic coupling may be used, wherein the distal portion of the extender element may have a fixed polarity, such as to exert a distally acting force on the cleaning unit. More specifically, as the distal portion of the extender element moves in the distal direction, it repels the cleaning unit such that the arms of the cleaning unit are pressed against the distal walls of the respective apertures. As the distal portion moves away, the cleaning unit returns to its rest position due to the elasticity of the arms, as explained above.

According to some embodiments, not depicted in the figures, catheter system 1300 includes an occlusion sensor, which may be similar to sensor 718 of catheter system 600, and which is functionally associated with PCB 1480, respectively.

As used herein, according to some embodiments, the terms “motion actuator”, “motion generator” and “vibration generator” are used interchangeably.

As used herein, according to some embodiments, the terms “tube distal section” and “catheter distal section” may be used interchangeably.

As used herein, according to some embodiments, the terms “extender” and “extender element” are used interchangeably.

The skilled person will understand that when referring to computational functions as being “performed” by PCB 780, it is actually electronic/control/processing circuitry included in PCB 780, which performs these functions.

The skilled person will understand that when stating, for example, that “the power to the cleaning unit is increased”, according to some embodiments, what is meant is that the power supplied to the motion generator is increased (e.g. the power supplied to the coil, of the electromagnet, so as to induce motion of the magnet (of the electromagnet) which may form a part of the cleaning unit).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1-36. (canceled)

37. A self-cleaning catheter system for fluid passage, the self-cleaning catheter system comprising:
a catheter configured to be implanted in a body cavity of a subject, the catheter comprising at least one aperture fluidly coupling the catheter to the outside thereof;
a cleaning unit configured for motion in the catheter such as to at least one of mechanically prevent, remove, and mitigate occlusion in at least one of the at least one aperture;
an implantable controller; and
at least one sensor communicatively associated with the implantable controller;
wherein the cleaning unit is functionally associated with the implantable controller; and
wherein the implantable controller is configured to (i) receive at least one signal from the at least one sensor, the at least one signal being indicative of a state of occlusion in the catheter, and at least one of (ii) provide an indication of the state of occlusion if the at least one signal indicates at least a partial blockage in the catheter and (iii) activate the cleaning unit if the at least one signal indicates at least a partial blockage of the catheter.

38. The self-cleaning catheter system of claim 37, wherein the at least one sensor comprises one or more of a pressure sensor configured to measure pressure within at least one of the catheter and the body cavity, and a flow meter configured to measure fluid-flow rate in the catheter, and
wherein the received at least one signal comprises at least one of a pressure related signal indicative of a pressure in at least one of the body cavity and the catheter and a fluid-flow related signal indicative of a fluid flow rate through the catheter; and wherein at least one of the pressure being above an upper pressure threshold, and the fluid flow rate being below a flow rate threshold, is indicative of at least partial blockage in the catheter.

39. The self-cleaning catheter system of claim 38, wherein the body cavity comprises a brain ventricle and wherein the implantable controller is configured to be implanted in the head of the subject outside the skull and beneath the skin, and wherein the pressure-related signal is indicative of intracranial pressure,

40. The self-cleaning catheter system of claim 37, wherein the catheter further comprises a catheter tube and a catheter tip member, which is distally positioned and fluidly connected to the catheter tube, wherein the catheter tip member comprises one or more of the at least one aperture, and wherein the catheter tip member at least partially houses the cleaning unit.

41. The self-cleaning catheter system of claim 40, wherein the cleaning unit comprises an elongated shaft comprising at least one arm configured to project into the at least one aperture and to move therein; and
wherein the motion of the cleaning unit in the catheter comprises vibration and the movement of the at least one arm within the at least one aperture is induced by the vibration of the cleaning unit.

42. The self-cleaning catheter system of claim 41, further comprising a vibration generator functionally associated with the implantable controller and configured to induce the vibration of the cleaning unit.

43. The self-cleaning catheter system of claim 42, wherein the vibration generator is an electromagnet and wherein the cleaning unit comprises or is mechanically coupled to a magnet of the electromagnet.

44. The self-cleaning catheter system of claim 37, wherein the at least one sensor is configured to be activated either on a periodic basis or continuously or substantially continuously.

45. The self-cleaning catheter system of claim 37, wherein the at least one sensor is housed in the catheter or embedded in a wall of the catheter.

46. The self-cleaning catheter system of claim 37, wherein the catheter is fluidly connected to a valve and/or a pump for evacuating fluid from the catheter, wherein the valve and/or the pump are functionally associated with the implantable controller, which is configured to open and close the valve and/or to switch the pump on and off; and
wherein the at least one sensor comprises at least two sensors: a first sensor, positioned in the catheter tip member, and a second sensor, positioned in, on, or in proximity to the valve and/or the pump, the second sensor being configured to measure pressure and/or fluid-flow rate at or in proximity to the valve and/or the pump.

47. The self-cleaning catheter system of claim 37, further comprising an implantable power receiver configured for receiving wireless power transfer (WPT) from an external activation unit, the implantable power receiver being further configured to at least partially power the catheter system, and
wherein the indication of the state of occlusion is configured to be transmitted to the external activation unit by either the implantable power receiver or a communication unit of the implantable controller, and wherein the external activation unit is configured to trigger an alert when the indication of the state of occlusion indicates at least partial blockage in the catheter.

48. The self-cleaning catheter system of claim 47, wherein the implantable controller is configured to prevent the activation of the cleaning unit if at least one of the power received by the implantable power receiver is above an upper power threshold and the power received by the implantable power receiver does not originate from the external activation unit.

49. The self-cleaning catheter system of claim 37, further comprising an implantable power source configured to at least partially power the self-cleaning catheter system.

50. The self-cleaning catheter system of claim 49, wherein the implantable controller comprises a communication unit configured to transmit the indication of the state of occlusion to an external controller, the external controller being configured to generate an alert when the indication of the state of occlusion indicates at least partial blockage in the catheter.

51. A kit for fluid passage in a body cavity of a subject, the kit comprising:
a self-cleaning catheter system comprising:
a catheter configured to be implanted in a body cavity of a subject, the catheter comprising at least one aperture fluidly coupling the catheter to the outside thereof;
a cleaning unit configured for motion in the catheter such as to at least one of mechanically prevent, remove, and mitigate occlusion in at least one of the at least one aperture;
an implantable controller;
an implantable power receiver;
at least one sensor communicatively associated with the implantable controller; and
an external activation unit configured for powering the self-cleaning catheter system, the external activation unit comprising a power transmitter and a processing circuitry functionally associated with the power transmitter;
wherein the power transmitter is configured for wireless power transfer (WPT) to the implantable power receiver of the catheter system when the catheter system is implanted in the body cavity of the subject.

52. The kit of claim 51, wherein the cleaning unit is functionally associated with the implantable controller and the implantable controller is configured to (i) receive at least one signal from the at least one sensor, the at least one signal being indicative of a state of occlusion in the catheter, and at least one of: (ii) provide an indication of the state of occlusion if the at least one signal indicates at least a partial blockage in the catheter and (iii) activate the cleaning unit if the at least one signal indicates at least a partial blockage of the catheter.

53. The kit of claim 51, wherein the external activation unit is further configured for placement on, to be attached to, worn on, and/or to be held against a body part of the subject, such as to enable the WPT from the power transmitter to the implantable power receiver, wherein the body part comprises the body cavity.

54. The kit of claim 53, wherein the body cavity comprises a brain ventricle, the fluid comprises cerebrospinal fluid, and wherein the external activation unit comprises a headpiece, or is configured to be mounted on a headpiece, the headpiece being configured to be worn on the head of the subject.

55. The kit of claim 54, wherein the processing circuitry of the external activation unit is configured to at least one of automatically initiate WPT to the implantable power receiver when the headpiece is positioned on the head of the subject in a predetermined position and prevent WPT to the implantable power receiver if the headpiece is not positioned on the head of the subject in the predetermined position.

56. The kit of claim 1515, wherein the external activation unit comprises, or is configured to be connected to, at least one feedback component configured to output one or more feedback signals indicating one or more of: that the power transmitter is transferring power to the implantable power receiver, that a cleaning session is being effected, and that a cleaning session has terminated; and
wherein the at least one feedback component comprises one or more of: a speaker configured to output one or more audio signals, and a visual component configured to output one or more visual signals.

57. The kit of claim 51, wherein the external activation unit is communicatively associated with a mobile communication device, and wherein the external activation unit is further configured to be controlled using software installable on the mobile communication device
